(19) United States
(12) Patent Application Publication
(10) Pub. No.: US 2017/0087224 A1
(43) Pub. Date: Mar. 30, 2017

(54) DELIVERY METHODS AND COMPOSITIONS

(71) Applicant: Agenovir Corporation, South San Francisco, CA (US)

(72) Inventor: Stephen R. Quake, Stanford, CA (US)

(21) Appl. No.: 15/277,595

(22) Filed: Sep. 27, 2016

Related U.S. Application Data
(60) Provisional application No. 62/234,340, filed on Sep. 29, 2015.

(51) Int. Cl. A61K 38/46; A61M 37/00; A61N 1/32; A61K 9/00; A61K 47/48

(52) U.S. Cl. CPC A61K 38/465; A61K 9/0009; C12Y 301/00; A61K 47/48092; A61N 1/327; A61M 37/0092; A61K 48/00

(57) ABSTRACT

The invention provides methods and compositions that remove target genetic material from a subject by delivery of an enzyme that degrades the target genetic material. The methods include delivering a composition of a nucleic acid to a tissue, such as skin, of a subject along with various types of energy to enhance permeability of the tissue and cause the nucleic acid to enter cells of the tissue, wherein the nucleic acid comprises a gene for an enzyme that cuts target genetic material. The nucleic acid may be a plasmid comprising a cas9 gene and at least one gene for a short guide RNA (sgRNA) and the target genetic material may be viral genome, i.e., with the sgRNA complementary to a portion of the viral genome.

### RELATED APPLICATION

This application claims the benefit of U.S. Provisional Patent Application Ser. No. 62/234,340, filed Sep. 29, 2015, incorporated by reference.

### TECHNICAL FIELD

The invention generally relates to methods of therapy delivery.

### BACKGROUND

Viral infections such as hepatitis, HIV, and the herpes family of viruses (herpesviridae) can affect infected individuals in ways ranging from social embarrassment to death. These viruses can establish latent infections that lie dormant in a subject for a long time in what is called viral latency. Latency is a period in the viral life cycle in which, after initial infection, viral proliferation ceases. However, the viral genome is not fully eradicated. As a result, the virus can reactivate, causing acute infection and producing large amounts of progeny without any new infection and complicating treatment of the aforementioned viruses.

Certain promising methods of viral treatment includes the use of gene editing systems to target and remove viral genomic material from infected cells. Gene editing systems include the use of Clustered Regularly Interspace Short Palindromic Repeat (CRISPR) associated endonuclease and guide RNAs complementary to virus-specific target sequences. These gene editing systems are currently administered via methods such as hypodermic injection, inhalation, or transmucosal or peroral delivery. These other methods are often painful and invasive and may provide a more circuitous route to the target cells which can lead to gastrointestinal symptoms or other side effects as well as modification and degradation of the therapeutic composition before it can reach the target cells. Introducing sufficient amounts of a therapeutic gene editing system to a target tissue and then into the targeted cells themselves remains a challenge.

### SUMMARY

The invention provides methods and systems for targeted genomic alteration. The present invention addresses the challenges of producing and delivering into host cells, a composition such as a programmable nuclease capable of specifically degrading target genetic material, such as a viral genome, without affecting the host's own genetic material or viability of the host cell. Methods of the invention include techniques for enhancing transport of compositions into tissue and through individual cellular membranes through the co-administration of energy to the target cell or tissue.

Transdermal or transmucosal delivery provides numerous advantages over other methods of delivery. Specifically, transdermal or transmucosal administration can provide more direct, relatively painless entry into cells of the body and generally produces fewer side effects than other administration methods. Such delivery can also provide other benefits such as enabling home application and timed release of a compound.

While transdermal delivery is a promising avenue for drug administration, it also poses its own set of challenges. One function of skin tissue is to provide a barrier between the body and the outer environment. Accordingly, skin contains barrier layers, such as the stratum corneum, which can make it difficult to pass therapeutic compounds into the body through the skin. The present invention relates to several energy-mediated delivery methods in which energy is administered to the skin, liver, or other tissue in order to increase permeability of the tissue and control uptake of the therapeutic compound by the tissue. These delivery methods may include one or more of the following, ultrasound mediated delivery (both high and low frequency or cavitational or no-cavitational), iontophoretic transdermal delivery, electroporation, chemical mediated delivery, thermal ablation of the stratum corneum, magnetophoresis, photomechanical waves, and mechanical methods such as microdermabrasion, gene guns, and microneedles. Of particular interest are transcellular delivery methods as opposed to intercellular delivery methods as the transcellular methods include passage through cellular membranes and may be used for transfection. In certain embodiments, genetic material may be transported across the cellular membrane by engineered proteins which are themselves introduced into the body through transdermal methods described herein.

Using the above or other delivery methods, a composition such as a programmable nuclease or a vector encoding the same is delivered to the cells. Where the vector is nucleic acid such as a plasmid encoding a programmable nuclease, expression of the nuclease allows it to degrade or otherwise interfere with the target genetic material.

In certain aspects, the invention provides a kit for delivering an antiviral therapy. The kit includes a device operable to apply energy to tissue; and a nucleic acid encoding a programmable nuclease that has been programmed to cleave a target in genetic material of a virus.

In certain embodiments, the device is an electroporation device comprising an electroporation generator and at least one electrode. The programmable nuclease is an RNA-guided nuclease. The kit may include an elongate member with an inner lumen, wherein said inner lumen is configured for delivery of the nucleic acid to a treatment site within a subject. The at least one electrode may be coated with the nucleic acid. Optionally, the nucleic acid encoding the programmable nuclease is mRNA encoding the programmable nuclease and is encapsulated in a nanoparticle (e.g., of lipids). The RNA-guided nuclease may be CasO.

In some embodiments, the device comprises an ultrasonic transducer; the nucleic acid is mRNA encoding the programmable nuclease; the kit includes an elongate member (e.g., needle) with an inner lumen, wherein said inner lumen is configured for delivery of the nucleic acid to a treatment site within a subject, or combinations thereof. Preferably, the nucleic acid is provided within microbubbles within the elongate member. Optionally, the programmable nuclease is Cas and the microbubbles further include one or more guideRNA. The ultrasonic transducer operates to provide low-intensity, non-cavitational ultrasound.

Aspects of the invention provide a kit for delivering an antiviral therapy. The kit includes a device operable to apply energy to tissue; and a programmable nuclease that has been programmed to cleave a target in genetic material of a virus.

In certain embodiments, the device is an electroporation device comprising an electroporation generator and at least one electrode. The programmable nuclease is an RNA-guided nuclease (e.g., Cas9) complexed with a guide RNA as an active ribonucleoprotein (RNP), wherein the guide RNA is complementary to a target within viral genetic material and is not complementary to any target within a human genome. The kit may include an elongate member with an inner lumen, wherein said inner lumen is configured for delivery of the RNP to a treatment site within a subject. Preferably, the RNP is encapsulated in a nanoparticle.

In some embodiments, the kit includes an ultrasonic transducer. The programmable nuclease may be an RNA-guided nuclease complexed with a guide RNA as an active ribonucleoprotein (RNP), wherein the guide RNA is complementary to a target within viral genetic material and is not complementary to any target within a human genome. The kit may include an elongate member with an inner lumen, wherein said inner lumen is configured for delivery of the nucleic acid to a treatment site within a subject. Optionally, the RNP is provided within microbubbles within the elongate member. Preferably, the ultrasonic transducer operates to provide low-intensity, non-cavitational ultrasound.

Any suitable programmable nuclease may be delivered using any kit or method of the invention and may be delivered in active form (e.g., as a protein or ribonucleoprotein (RNP)), encoded in messenger RNA, or encoded as a gene, e.g., on a nucleic acid vector such as a plasmid or viral vector. The programmable nuclease may be, for example, be an RNA-guided nuclease (e.g., a CRISPR-associated nuclease, such as Cas9 or a modified Cas9 or Cpf1 or modified Cpf1). The programmable nuclease may be a TALEN or a modified TALEN or a zing finger nuclease (ZFN). In certain embodiments, the programmable nuclease may be a DNA-guided nuclease (e.g., a Pyrococcus furiosus Argonaute (PfAgo) or Natronobacterium gregoryi Argonaute (NgAgo). The programmable nuclase may be a high-fidelity Cas9 (hi-fi Cas9), e.g., as described in Kleinstiver et al., 2016, High-fidelity CRISPR-Cas9 nucleases with no detectable genome-wide off-target effects, Nature 529:490-495, incorporated by reference. Where the programmable nuclease is, e.g., an RNA-guided nuclease and delivered via nucleic acid vector, the nucleic acid may contain guide RNAs that target the nuclease to the target genetic material. Where the target genetic material includes the genome of a virus, guide RNAs complementary to parts of that genome can guide the degredation of that genome by the nuclease, thereby preventing any further replication or even removing any intact viral genome from the cells entirely. By these means, latent viral infections can be targeted for eradication. Since methods for gene delivery of nuclease with activity specific to the genome of a latent virus are provided, methods of the invention may be used to address latent viral infections. Thus methods and compositions of the invention may provide relief from the adverse consequences of viruses such as HBV, Epstein-Barr, or others.

In certain aspects, the invention provides methods for removing target genetic material from a subject. The methods include delivering a composition to tissue and applying energy to the tissue to increase permeability of the tissue and facilitate the composition to enter the tissue or even the cells of the tissue. The composition includes a programmable nuclease or nucleic acid encoding the same. For example, the composition may include an active Cas9 RNP or a plasmid or mRNA encoding Cas9.

The applied energy may be high intensity focused ultrasound. The applied energy may alternatively be low frequency ultrasound. In certain embodiments, the energy may be applied through electroporation. The energy may be applied through iontophoresis. In some embodiments, the applied energy may be thermal. The energy may be applied through radio waves. The energy may be applied mechanically through microneedles, or microdermabrasion, or by using a gene gun to bombard the cells. In certain embodiments, the energy may be applied through a magnetic field. The energy may be applied through photomechanical waves. In various embodiments, the solution may be delivered transdermally.

The nucleic acid may be a plasmid comprising a cas9 gene and at least one gene for a short guide RNA (sgRNA) and the target genetic material may be viral genome, i.e., with the sgRNA complementary to a portion of the viral genome. In some embodiments, the viral genome is a hepatitis B genome and the plasmid contains genes for one or more sgRNAs targeting locations in the hepatitis B genome such as PreS1, DR1, DR2, a reverse transcriptase (RT) domain of polymerase, an Hbx, the core ORF, or combinations thereof.

In certain embodiments, the target genetic material is genome of a virus and the nucleic acid is a plasmid comprising a cas9 gene and at least one sgRNA targeting the genome of the virus. The plasmid further includes a viral origin of replication (i.e., such that prospective replication of the latent virus leads to replication of the very plasmid genes targeting that virus). In an exemplary embodiment, the virus is hepatitis B and the sgRNA includes one or more of sgHBV-RT, sgHBV-Hbx, sgHBV-Core, and sg-HBV-PerS1.

The nucleic acid may, in certain embodiments, include mRNA comprising a 5′ cap. In various embodiments, the enzyme may be a transcription activator-like effector nuclease (TALEN).

In certain aspects, the invention provides a method for disrupting target genetic material from a subject. The method includes delivering a composition comprising a ribonucleoprotein to a tissue by applying an energy to the tissue to increase permeability of the tissue and allow the nucleic acid to enter cells of the tissue, wherein the ribonucleoprotein comprises an enzyme that cuts target genetic material and at least one short guide RNA (sgRNA). The enzyme may be Cas9 or a TALEN. The energy may be applied through electroporation or may be ultrasound. The applied energy may be low frequency ultrasound or high intensity focused ultrasound. The energy may be applied through iontophoresis. In some embodiments, the applied energy may be thermal. The energy may be applied through radio waves. The energy may be applied mechanically through microneedles, or microdermabrasion, or by using a gene gun to bombard the cells. In certain embodiments, the energy may be applied through a magnetic field. The energy may be applied through photomechanical waves. In various embodiments, the solution may be delivered transdermally.

The target genetic material may be viral, i.e., with the sgRNA complementary to a portion of the viral genome. In some embodiments, the viral genome is a hepatitis B genome and the one or more sgRNAs target locations in the hepatitis B genome such as PreS1, DR1, DR2, a reverse transcriptase (RT) domain of polymerase, an Hbx, the core ORF, or combinations thereof.

### BRIEF DESCRIPTION OF THE DRAWINGS

FIG. 1 diagrams a method for removing target genetic material from a subject.

FIG. 2 shows key parts in the HBV genome targeted by CRISPR guide RNAs.

FIG. 3 shows a gel resulting from an in vitro CRISPR assay against HBV.

FIG. 4 diagrams a plasmid according to certain embodiments.

FIG. 5 shows a system, including an ultrasound transducer, for removing target genetic material from a subject according to certain embodiments.

FIG. 6 shows a system, including an electroporation device, for removing target genetic material from a subject according to certain embodiments.

FIG. 7 shows a system, including a gene gun, for removing target genetic material from a subject according to certain embodiments.

FIG. 8 shows a system, including a iontophoresis device, for removing target genetic material from a subject according to certain embodiments.

FIG. 9 shows a system, including a microneedle patch, for removing target genetic material from a subject according to certain embodiments.

FIG. 10 shows a system, including a microdermabrader, for removing target genetic material from a subject according to certain embodiments.

FIG. 11 shows a system, including a thermal ablation device, for removing target genetic material from a subject according to certain embodiments.

FIG. 12 shows a system, including a magnetic drug delivery system, for removing target genetic material from a subject according to certain embodiments.

FIG. 13 shows a system, including a laser, for removing target genetic material from a subject according to certain embodiments.

FIG. 14 shows a process for assessing the effect of a Cas9/HPV 16-specific sgRNA ribonucleic protein (RNP) on HPV-16+ cells.

FIG. 15 shows target locations for various sgRNAs along the E6 and E7 genes of HPV-16.

FIG. 16 illustrates HPV-16+ cell counts after introduction by electroporation of RNPs with various sgRNAs with targets along HPV-16 E6 and E7 genes.

FIG. 17 illustrates target locations and quantitative PCR (qPCR) primer locations on the E6 and E7 genes of HPV-16.

FIG. 18 shows qPCR results focusing on the E6 and E7 genes 1 and 2 days after treatment with various HPV 16-specific RNPs.

FIG. 19 shows viable cell counts 1 and 6 days after treatment with various HPV 16-specific RNPs.

FIG. 20 shows a process for assessing the effect of a HPV 16-specific sgRNA and mRNA encoding Cas9 protein on HPV-16+ cells.

FIG. 21 shows normalized cell counts after 1, 3, and 6 days post-nucleofection with various Cas9 mRNA and sgRNA combinations.

FIG. 22 shows cell counts for cells treated with various sgRNA and a variety of Cas9 mRNA after 6 days.

FIG. 23 shows a process for assessing the effect of a Cas9/HPV 18-specific sgRNA ribonucleic protein (RNP) on HPV-18+ cells.

FIG. 24 shows target locations for various sgRNAs along the E6 gene of HPV-18.

FIG. 25 illustrates cell counts after introduction by electroporation of RNPs with various sgRNAs targeting the HPV-18 E6 gene.

FIG. 26 shows a viable cell count comparison for HPV-18+ cancer cells 5 days post electroporation with sgHPV18E6-2/Cas9 in RNP format or in mRNA/sgRNA format.

FIG. 27 shows a comparison of viable cell counts in mRNA and RNP treated cells by μg dose of Cas9 mRNA or protein.

FIG. 28 illustrates an HBV episomal DNA cell model.

FIG. 29 shows target locations on the HBV genome of various sgRNAs.

FIG. 30 shows results of gel electrophoresis separations indicating cleavage of HBV DNA in cells transduced with sgRT RNA, sgHBx RNA, sgCore RNA, and sgPreS1 RNA.

FIG. 31 shows HBV DNA quantity determined by qPCR in untreated cells and cells treated with HBV-specific sgRNAs and Cas9.

### DETAILED DESCRIPTION

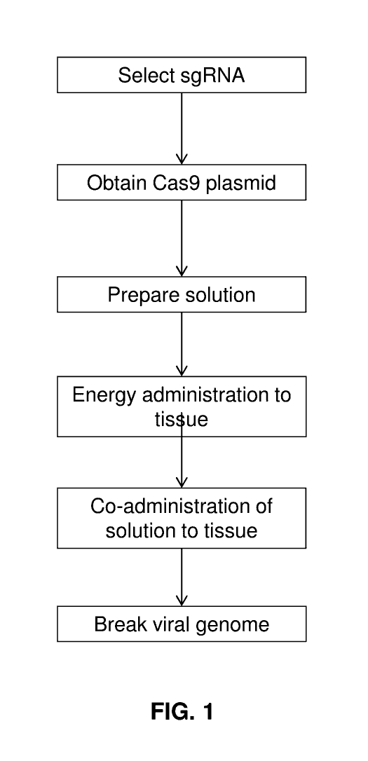

FIG. 1 diagrams a method for removing target genetic material from a subject. The method includes co-administering energy and a composition to a tissue, in order to cause the composition to enter cells of the tissue. The composition includes a programmable nuclease or nucleic acid encoding the same such as a plasmid or mRNA. The programmable nuclease is an enzyme that has been programmed to target and cleave genetic material.

Any suitable programmable nuclease may be used. Programmable nucleases include zinc-finger nucleases (ZFNs), transcription activator-like effector nucleases (TALENs) and RNA-guided nucleases such as the bacterial clustered regularly interspaced short palindromic repeat (CRISPR)-Cas (CRISPR-associated) nucleases or Cpf1. Programmable nucleases also include DNA-guided nuclease (e.g., a Pyrococcus furiosus Argonaute (PfAgo) or Natronobacterium gregoryi Argonaute (NgAgo). The programmable nuclase may be a high-fidelity Cas9 (hi-fi Cas9), e.g., as described in Kleinstiver et al., 2016, High-fidelity CRISPR-Cas9 nucleases with no detectable genome-wide off-target effects, Nature 529:490-495, incorporated by reference.

Zinc finger nuclease (ZFN), transcription activator-like effector nuclease (TALEN), and clustered regularly interspaced short palindromic repeats (CRISPR), have provided great promise to gene therapy (Cell Stem Cell. 2013, 13(6): 653-8). By targeting viral DNA, recent studies demonstrated the treatment of latent viral infections in human cells with CRISPR. See Wang & Quake, 2014, RNA-guided endonuclease provides a therapeutic strategy to cure latent herpesviridae infection, PNAS 111(36):13157-13162 and Hu et al., 2014, RNA-directed gene editing specifically eradicates latent and prevents new HIV-1 infection, PNAS 111(31):11461-6, both incorporated by reference. Methods and materials of the present invention may be used to apply targeted endonuclease to specific genetic material such as a latent viral genome like HBV. The invention further provides for the efficient and safe delivery of nucleic acid (such as a DNA plasmid) into target cells (e.g., hepatocytes).

In an exemplary embodiment, the invention provides a combination one or more oof the gene delivery methods described herein and targeted endonuclease to treat a viral infection.

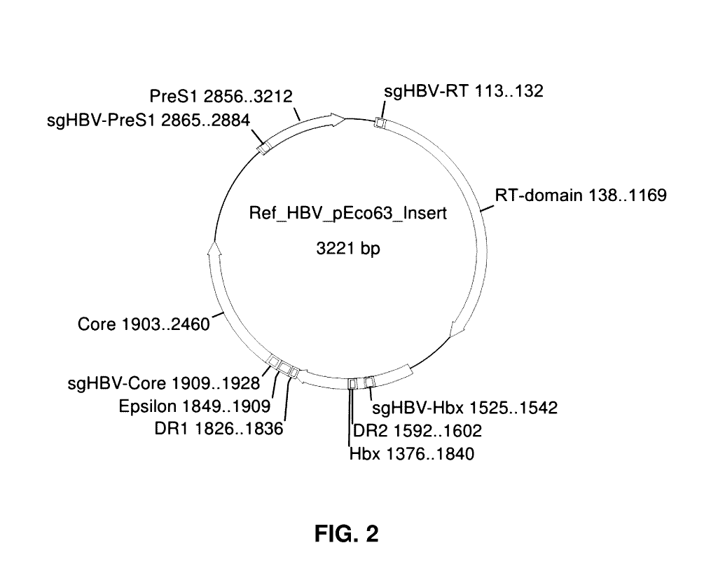

FIG. 2 diagrams the HBV genome. In some embodiments, the invention uses one or several guide RNAs against key features within a genome such as the HBV genome shown in FIG. 2. With reference to FIG. 2, HBV starts its infection cycle by binding to the host cells with PreS1. Guide RNA against PreS1 locates at the 5′ end of the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation. Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces.

FIG. 2 shows key parts in the HBV genome targeted by CRISPR guide RNAs.

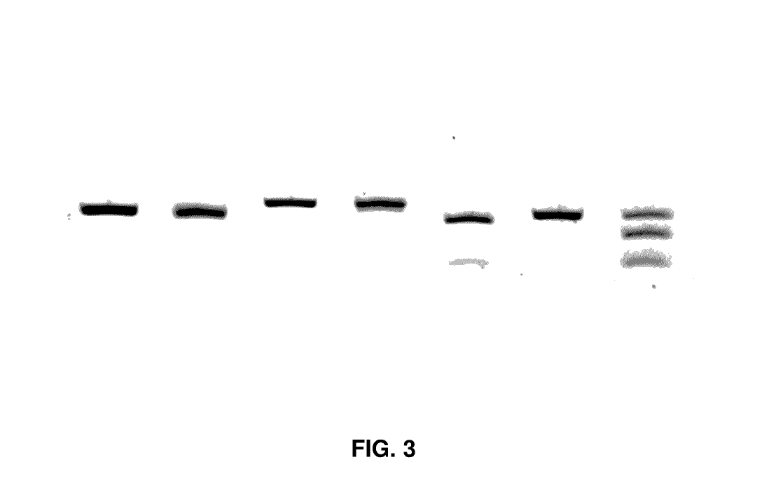

FIG. 3 shows a gel resulting from an in vitro CRISPR assay against HBV. Lane 1, 3, 6: PCR amplicons of HBV genome flanking RT, Hbx-Core, and PreS1. Lane 2, 4, 5, and 7: PCR amplicons treated with sgHBV-RT, sgHBV-Hbx, sgHBV-Core, sgHBV-PreS1.

The invention provides the aforementioned guide RNAs. To demonstrate, an in vitro assay was performed with cas9 protein and DNA amplicons flanking the target regions. As shown in FIG. 2, DNA electrophoresis shows strong digestion at the target sites.

To achieve the CRISPR activity in cells, expression plasmids coding cas9 and guide RNAs are delivered to cells of interest (e.g., cells carrying HBV DNA). To demonstrate in an in vitro assay, anti-HBV effect may be evaluated by monitoring cell proliferation, growth, and morphology as well as analyzing DNA integrity and HBV DNA load in the cells.

To deliver the Cas9 and sgRNAs, the invention provides for the use various methods to increase permeability of the target tissue and control uptake of the therapeutic compound. These delivery methods may include one or more of the following, ultrasound mediated delivery (both high and low frequency or cavitational or no-cavitational), iontophoretic transdermal delivery, electroporation, chemical mediated delivery, thermal ablation of the stratum corneum, magnetophoresis, photomechanical waves, and mechanical methods such as microdermabrasion and microneedles. See Prausnitz & Langer, Transdermal drug delivery, Nature Biotechnology 26, 1261-1268 (2008), the contents of which are incorporated herein in their entirety for all purposes. Many of the above methods include applications in transdermal delivery across the stratum corneum as well as delivery across intracellular delivery by inducing cell membrane fluidity and allowing nucleic acid compositions of the invention to pass into cells.

In various embodiments, energy may delivered to cells or tissue through ultrasound waves. See Smith, Perspectives on transdermal ultrasound mediated drug delivery, Int J Nanomedicine. 2007 December; 2(4): 585-594, the contents of which are incorporated herein in their entirety for all purposes. These methods are sometimes referred to a sonophoresis or phonophoresis. Ultrasound mediated transdermal drug delivery may be used with a range of ultrasound frequencies and is generally categorized as high frequency (e.g., around 1-3 MHz) or low frequency (e.g., around 20 kHz). Ultrasound mediated transdermal drug delivery is sometimes divided into cavitational and noncavitational methods. Low frequency ultrasound is generally more effective at enhancing transdermal drug transport through cavitation induced bilayer disordering of the stratum corneum. Id. The permeability effects of cavitational bubbles generated in the stratum corneum through low frequency ultrasound may last for many hours. Prausnitz, 2008.

Ultrasound may be used to facilitate passage of compounds across cellular membranes in the form of encapsulated ultrasound microbubbles in any tissue. See Nozaki, et al., Enhancement of ultrasound-mediated gene transfection by membrane modification, The Journal of Gene Medicine, Vol. 5, Issue 12, pp. 1046-1055, December 2003; Liu, et al., Encapsulated ultrasound microbubbles: Therapeutic application in drug/gene delivery, Journal of Controlled Release, Vol. 114, Issue 1, 10 Aug. 2006, pp. 89-99; the contents of each which are incorporated herein in their entirety and for all purposes. Low-intensity ultrasound in combination with microbubbles has recently acquired much attention as a safe method of gene delivery. Ultrasound shows tissue-permeabilizing effect. It is non-invasive and site-specific. Ultrasound-mediated microbubbles have been proposed as an innovative method for noninvasive delivery of drugs and nucleic acids to different tissues. In ultrasound-triggered drug delivery, tissue-permeabilizing effect can be potentiated using ultrasound contrast agents, gas-filled microbubbles. The use of microbubbles for delivery of nucleic acids is based on the hypothesis that destruction of DNA-loaded microbubbles by a focused ultrasound beam during their microvascular transit through the target area will result in localized transduction upon disruption of the microbubble shell while sparing non-targeted areas. See Tsutsui et al., 2004, The use of microbubbles to target drug delivery, Cardiovasc Ultrasound 2:23, the contents of which are incorporated by reference.

Small, lipophilic compounds may be delivered with noncavitational ultrasound but success is limited with other, larger compounds. Heat has been shown to enhance transdermal delivery of some compounds and one aspect of ultrasound mediated delivery is the generation of heat in the tissue by the ultrasound waves.

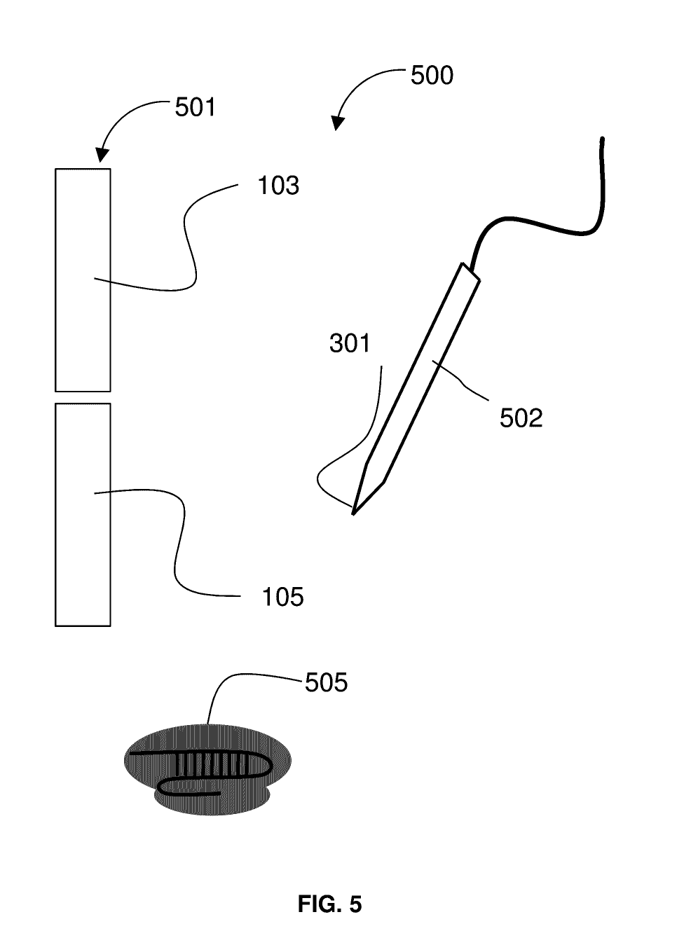

Ultrasound waves may be applied using single element or other known types of transducers such as those available from Blatek, Inc. (State College, Pa.). Thus, in some embodiments, the invention provides a system for treating a viral infection that includes an ultrasound transducer 301, a vector encoding a gene for an enzyme that cuts target genetic material such as Cas9 103, and a gRNA that targets a latent virus and that has no match in the human genome, as shown in FIG. 5.

FIG. 5 shows a kit 500 for delivering an antiviral therapy. The kit 500 has a device that includes an ultrasonic transducer 301 and is operable to apply energy to tissue; and either: a nucleic acid 501 with a gene 103 encoding a programmable nuclease that has been programmed to cleave a target in genetic material of a virus; or a programmable nuclease that has been programmed to cleave a target in genetic material of a virus. In certain embodiments, the nucleic acid 501 is mRNA encoding the programmable nuclease.

Optionally, the kit 500 may include one more guide RNA 105, which preferably hybridizes to a target in a viral genome is not complementary to a human genome. The kit may include an elongate member 502 with an inner lumen, wherein said inner lumen is configured for delivery of the nucleic acid to a treatment site within a subject. The nucleic acid may provided within microbubbles within the elongate member.

In certain embodiments, the programmable nuclease is Cas9 and the microbubbles further include one or more guideRNA. The elongate member may be a needle. Preferably, the ultrasonic transducer 301 operates to provide low-intensity, non-cavitational ultrasound.

In alternative embodiments, the programmable nuclease is an RNA-guided nuclease complexed with a guide RNA as an active ribonucleoprotein (RNP) 505, wherein the guide RNA is complementary to a target within viral genetic material and is not complementary to any target within a human genome. the kit may include an elongate member 502 with an inner lumen, wherein said inner lumen is configured for delivery of the RNP 505 to a treatment site within a subject. Optionally the RNP 505 is encapsulated in a nanoparticle, which may include, for example, lipids. Preferably, the RNA-guided nuclease is Cas9.

In certain embodiments, transdermal delivery may be enhanced through electroporation of the skin tissue. See Prausnitz, et al., Electroporation of mammalian skin: A mechanism to enhance transdermal drug delivery, Proc. Natl. Acad. Sci. USA Vol. 90, pp. 10504-10508, November 1993, the contents of which are incorporated herein in their entirety for all purposes.

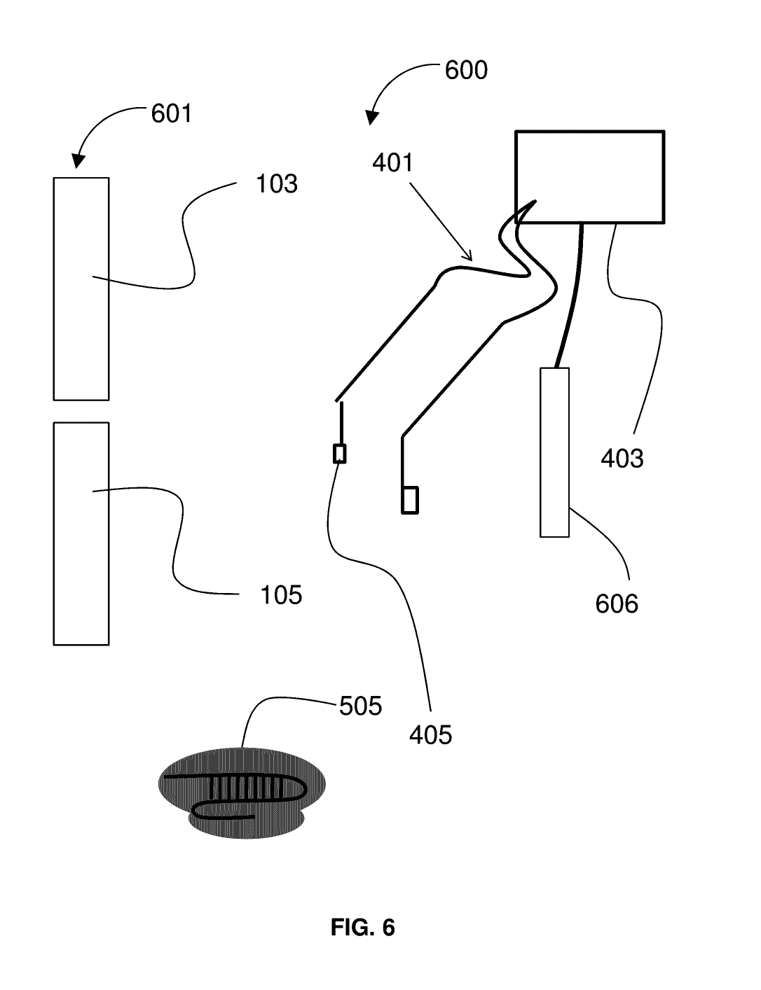

FIG. 6 shows a kit 600 for delivering an antiviral therapy. The kit 600 includes an electroporation device 401 operable to apply energy to tissue; and either (i) a nucleic acid encoding a programmable nuclease that has been programmed to cleave a target in genetic material of a virus; or (ii) a programmable nuclease that has been programmed to cleave a target in genetic material of a virus.

Preferably the electroporation device 401 comprising an electroporation generator 403 and at least one electrode 405. The programmable nuclease may be an RNA-guided nuclease.

The kit 600 may include an elongate member 606 (e.g., a needle) with an inner lumen, wherein said inner lumen is configured for delivery of the nucleic acid to a treatment site within a subject. Optionally, the at least one electrode 405 is coated with the nucleic acid. The nucleic acid encoding the programmable nuclease may be an mRNA 601 with a gene 103 encoding the programmable nuclease. The mRNA may be encapsulated in a nanoparticle, such as a lipid nanoparticle. Preferably, the RNA-guided nuclease is Cas9.

In certain embodiments, the programmable nuclease is an RNA-guided nuclease complexed with a guide RNA 105 as an active ribonucleoprotein (RNP) 505, wherein the guide RNA is complementary to a target within viral genetic material and is not complementary to any target within a human genome.

Electroporation involves the use of short, high-voltage pulses of electricity to reversibly disrupt cell membranes. Electroporation, like cavitational ultrasound, disrupts lipid bilayer structures in the skin, allowing for increased permeability and, accordingly, enhanced drug delivery. The electropores created through electroporation can persist for hours after treatment, and transdermal transport can be increased by orders of magnitude for small molecule drugs, peptides, vaccines and DNA. Side effects of electroporation, such as pain and muscle stimulation from the nerves below the stratum corneum layer, can be minimized through the use of closely spaced microelectrodes to constrain the electric field within the stratum corneum. Prausnitz, 2008.

Electroporation of cellular membranes can be used to increase cell membrane fluidity and allow passage of compounds into individual cells. See Ho, et al., Electroporation of Cell Membranes: A Review, Critical Reviews in Biotechnology, Vol. 16, Issue 4, 1996; Zhang, et al., Development of an Efficient Electroporation Method for Iturin A-Producing Bacillus subtilis ZK, Int. J. Mol. Sci. 2015, 16, 7334-7351; the contents of each which are incorporated herein in their entirety and for all purposes. Electroporation of cell membranes uses the same principles as described above with respect to transdermal applications. Id. As cell viability is essential to the methods of the invention, care must be taken in the application of the short high-voltage pulses.

Electroporation may be performed using an electroporation device 401 comprising, for instance, an electroporation generator 403 and electrodes 405 such as the Gemini X2 system available from Harvard Apparatus, Inc. (Holliston, Mass.). Thus, in some embodiments, the invention provides a system for treating a viral infection that includes electroporation device 401 comprising an electroporation generator 403 and electrodes 405, a vector encoding a gene for an enzyme that cuts target genetic material such as Cas9 103, and a gRNA that targets a latent virus and that has no match in the human genome, as shown in FIG. 6.

In various embodiments nucleic acid compositions of the invention may be introduced into host cells through biolistic transformation or particle bombardment using, for instance, a gene gun. See Gao, et al., Nonviral Gene Delivery: What We Know and What Is Next, AAPS J. 2007 March; 9(1): E92-E104; Yang, et al., In vivo and in vitro gene transfer to mammalian somatic cells by particle bombardment, Proc Natl Acad Sci USA, 1990; 87:9568-9572; the contents of each of which are incorporated herein in their entirety and for all purposes. Particle bombardment through a gene gun may be used, for example, to introduce compositions of the invention into cells of the skin, mucosa, or surgically exposed tissues within a confined area. In particle bombardment methods, nucleic acid is deposited on the surface of gold particles, which are then accelerated, for example, by pressurized gas, into cells or tissue such that the momentum of the gold particles carries the nucleic acid into the cells. Id.

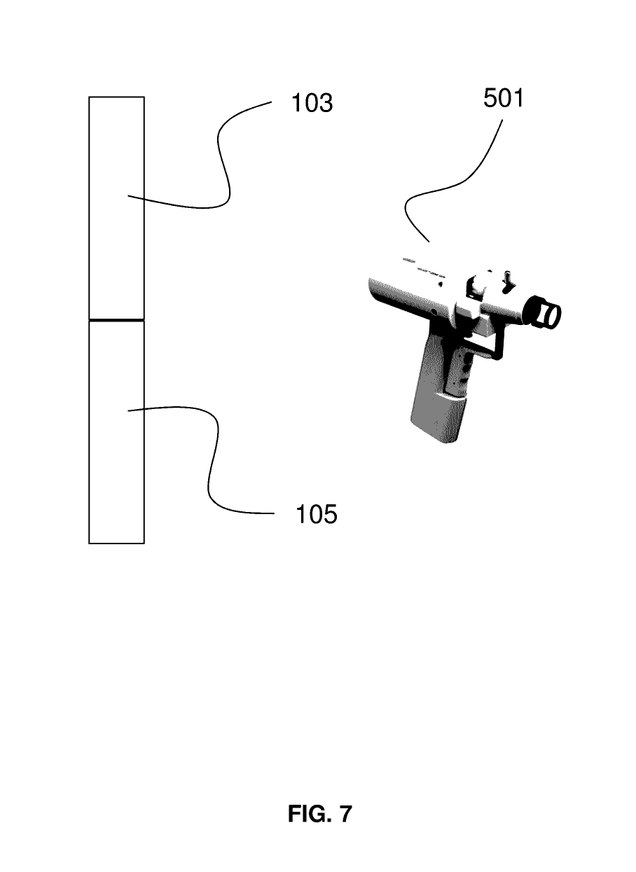

Particle bombardment may be performed using, for example a gene gun such as the Helios Gene Gun System available from Bio-Rad Laboratories, Inc. (Hercules, Calif.). Thus, in some embodiments, the invention provides a system for treating a viral infection that includes a gene gun 501, a vector encoding a gene for an enzyme that cuts target genetic material such as Cas9 103, and a gRNA that targets a latent virus and that has no match in the human genome, as shown in FIG. 7.

In various embodiments, transdermal delivery may be enhanced through iontophoresis. See Rawat, et al., Transdermal Delivery by Iontophoresis, Indian J Pharm Sci. 2008 January-February; 70(1): 5-10, the contents of which are incorporated herein in their entirety for all purposes. Iontophoresis includes application of a continuous low-voltage current to the skin to provide an electrical driving force for transport across the stratum corneum. Prausnitz, 2008. Therapeutic compounds having an electrical charge may be driven into the stratum corneum by creating a potential across the layer and applying the aforementioned current. One advantage of iontophoretic delivery is the ability to control the rate of drug delivery by altering the current level. Compounds without significant charge can be moved across the stratum corneum by electroosmotic flow of water generated by the movement of mobile cations (e.g., Na+) instead of fixed anions (e.g., keratin) in the stratum corneum. Id.

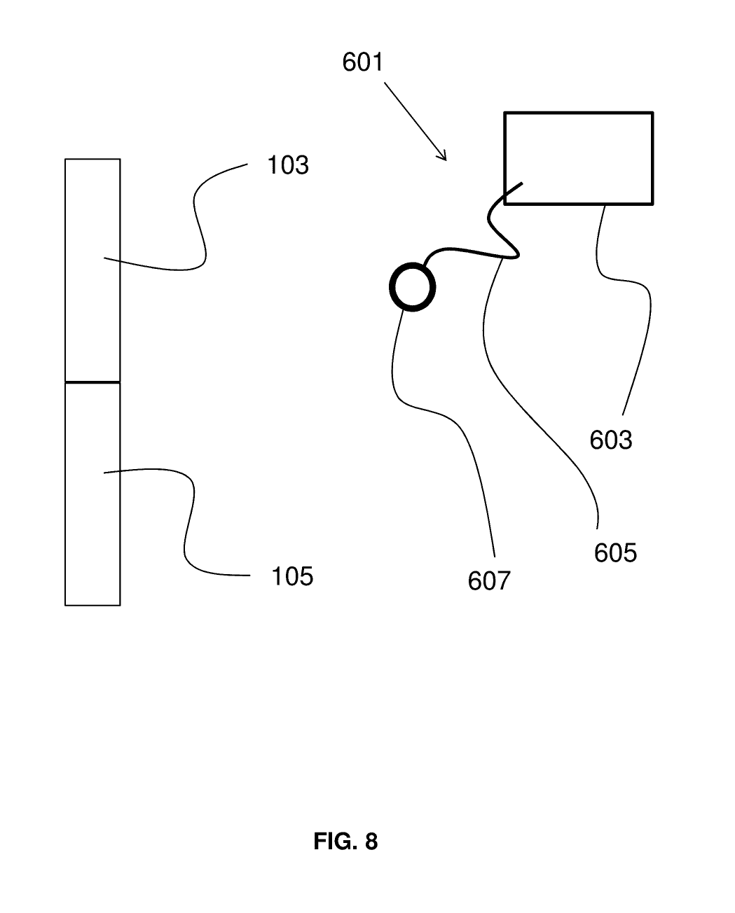

Iontophoresis may be performed using an iontophoresis device 601 comprising, for instance, an iontophoresis controller 603, leads 605 and conductive pads 607 such as the MIC2 Iontophoresis Controller and accessories available from Moor Instruments (Devon, United Kingdom). Thus, in some embodiments, the invention provides a system for treating a viral infection that includes an iontophoresis device 601 comprising, an iontophoresis controller 603, leads 605 and conductive pads 607, a vector encoding a gene for an enzyme that cuts target genetic material such as Cas9 103, and a gRNA that targets a latent virus and that has no match in the human genome, as shown in FIG. 8.

In certain embodiments, mechanical means of enhancing delivery of compounds into tissue may be used such as microdermabrasion or microneedles. Microneedles selectively permeabilize the stratum corneum by piercing it with very short needles. See Pausternitz, 2008. Microneedles have been shown to increase skin permeability to a variety of small molecules, proteins and nanoparticles and can be used in extended-release patches to control release of the compound into the skin. Id. Because the microneedles do not pierce to level of nerves within the skin tissue, the present a relatively painless means of enhancing transdermal drug administration. Compounds may also be coated on or encapsulated within microneedles and hollow also microneedles may also be used. Id. Microneedles enhance transdermal drug administration by creating micron-scale pathways into the skin and can also drive compounds into the skin when the microneedles themselves are coated with or encapsulate the compound. Id.

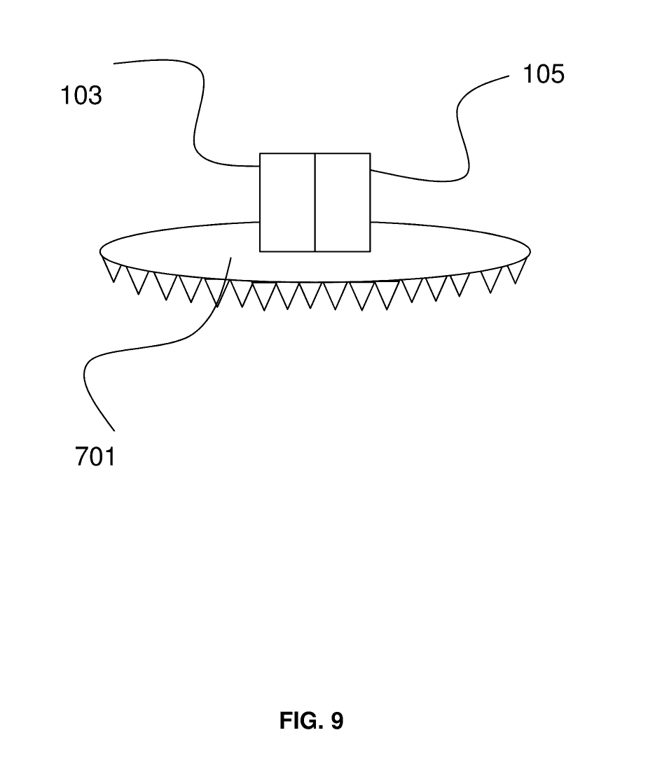

Microneedle patches 701 such as the solid microneedle patches available from 3M (Saint Paul, Minn.) may be used to deliver compositions of the invention. In certain embodiments, Hollow microneedle delivery systems such as the Hollow Microstructured Transdermal System available from 3M (Saint Paul, Minn.) may be used to deliver compositions of the invention. Thus, in some embodiments, the invention provides a system for treating a viral infection that includes a microneedle patch 701, a vector encoding a gene for an enzyme that cuts target genetic material such as Cas9 103, and a gRNA that targets a latent virus and that has no match in the human genome, as shown in FIG. 9.

Another mechanical method of transdermal administration contemplated by the invention is microdermabrasion. Microdermabrasion consists of ablating the stratum corneum through use of an abrasive. By physically removing that barrier to skin permeability, transdermal delivery of compounds is enhanced. See Prausnitz, 2008.

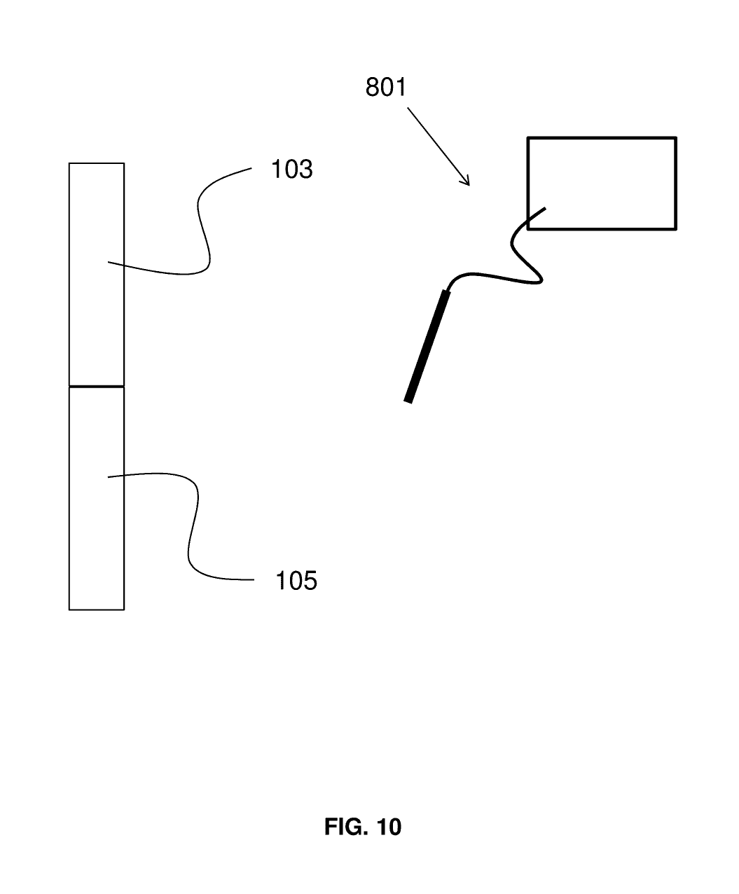

Microdermabrasion may be performed using a microdermabrader 801 such as the Ultrapeel Crystal available from Mattioli Engineering Corporation (McLean, Va.). Thus, in some embodiments, the invention provides a system for treating a viral infection that includes an microdermabrader 801, a vector encoding a gene for an enzyme that cuts target genetic material such as Cas9 103, and a gRNA that targets a latent virus and that has no match in the human genome, as shown in FIG. 10.

In certain embodiments thermal energy is applied to the tissue to enhance delivery of the nucleic acid to the tissue. See Parusnitz 2008. In one such method, thermal ablation, the skin surface is selectively heated to generate micron-scale perforations in the stratum corneum. Id. Heat may be applied in short, high intensity bursts to heat the tissue surface to hundreds of degrees for only microseconds or milliseconds. Id. These short bursts prevent propagation of the heat to deeper tissue which keeps the tissue viable and prevent pain for the patient. Id. The heat is used to vaporize water in the stratum corneum so that the expanding water creates micron-scale craters in the layer. Id. In various embodiments, heat may be generated through lasers or other optical means, radio waves (RF), ultrasound waves, or using electric current.

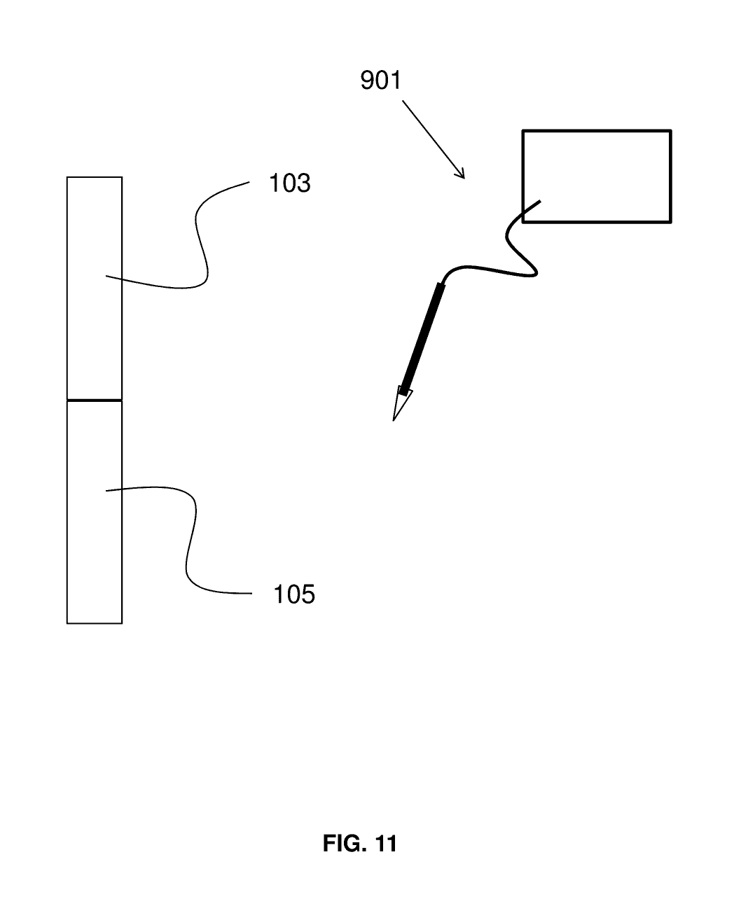

Thermal energy may be applied to tissue using, for example, a thermal ablation device such as the devices described in U.S. Pat. Pub. 2009/0318846 or in Lee, et al., Microsecond Thermal Ablation of Skin for Transdermal Drug Delivery, J Control Release. 2011 Aug. 25; 154(1): 58-68, the contents of each of which are incorporated herein in their entirety and for all purposes. Thus, in some embodiments, the invention provides a system for treating a viral infection that includes a thermal ablation device 901, a vector encoding a gene for an enzyme that cuts target genetic material such as Cas9 103, and a gRNA that targets a latent virus and that has no match in the human genome, as shown in FIG. 11.

Other methods of energy enhanced drug delivery useful in methods of the invention include magnetophoresis and the use of photomechanical waves. Magnetophoresis, or the use of magnetic fields to enhance transdermal drug delivery, does not appear to alter the permeability of the stratum corneum but instead acts to drive the compounds into the tissue through magnetokinesis, similar to the use of electric current in iontopohoresis. See Murthy, et al., Magnetophoresis for enhancing transdermal drug delivery: Mechanistic studies and patch design, Journal of Controlled Release, Volume 148, Issue 2, 1 Dec. 2010, Pages 197-203; U.S. Pat. Pub. No. 2002/0147424; the contents of each of which are incorporated herein in their entirety for all purposes. Magnetic nanoparticles may also be used to deliver nucleic acids of the invention across cellular membranes. Nucleic acid carriers can be responsive to both ultrasound and magnetic fields, i.e., magnetic and acoustically active lipospheres (MAALs). The basic premise is that therapeutic agents are attached to, or encapsulated within, a magnetic micro- or nanoparticle. These particles may have magnetic cores with a polymer or metal coating which can be functionalized, or may consist of porous polymers that contain magnetic nanoparticles precipitated within the pores. By functionalizing the polymer or metal coating it is possible to attach, for example, therapeutic nucleic acids of the invention to target viral genome within a host cell. The particle/therapeutic agent complex may be introduced into the body through any of the transdermal methods mentioned herein or through injection into the blood stream or other known methods. Magnetic fields are then introduced, generally from high-field, high-gradient, rare earth magnets, and are focused over the target site and the forces on the particles as they enter the field allow them to be captured and extravasated at the target. See Guo, et al., Recent Advances in Non-viral Vectors for Gene Delivery, Acc Chem Res. 2012 Jul. 17; 45(7): 971-979, the contents of which are incorporated herein in their entirety and for all purposes.

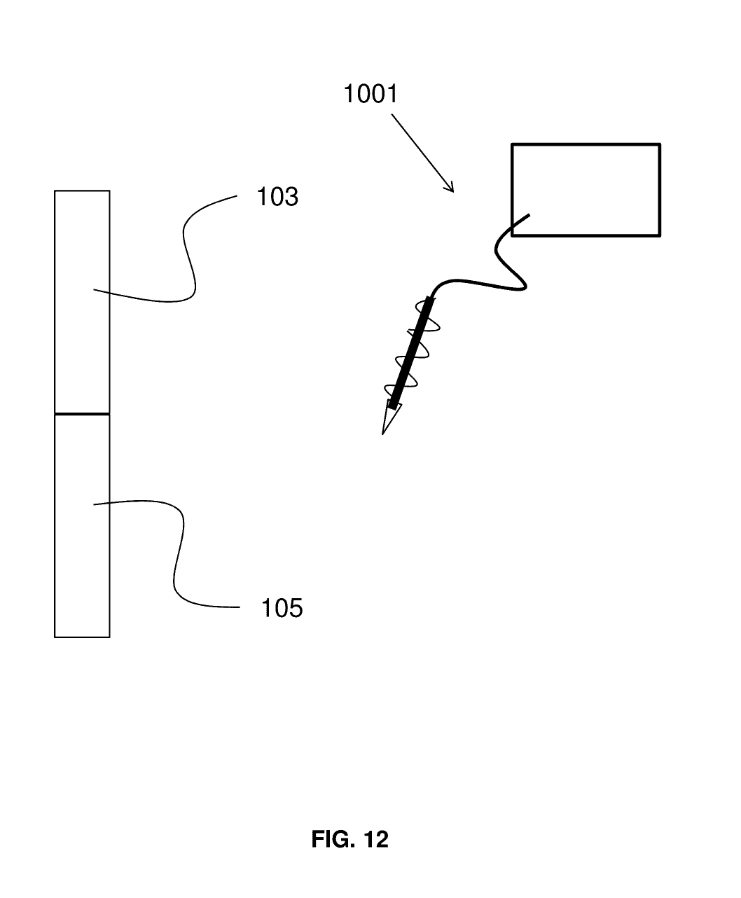

Magnetophoresis may be carried out using a magnetic drug delivery system such as described in US Pat. Pub. No. 2002/0147424. Thus, in some embodiments, the invention provides a system for treating a viral infection that includes a magnetic drug delivery system 1001, a vector encoding a gene for an enzyme that cuts target genetic material such as Cas9 103, and a gRNA that targets a latent virus and that has no match in the human genome, as shown in FIG. 12.

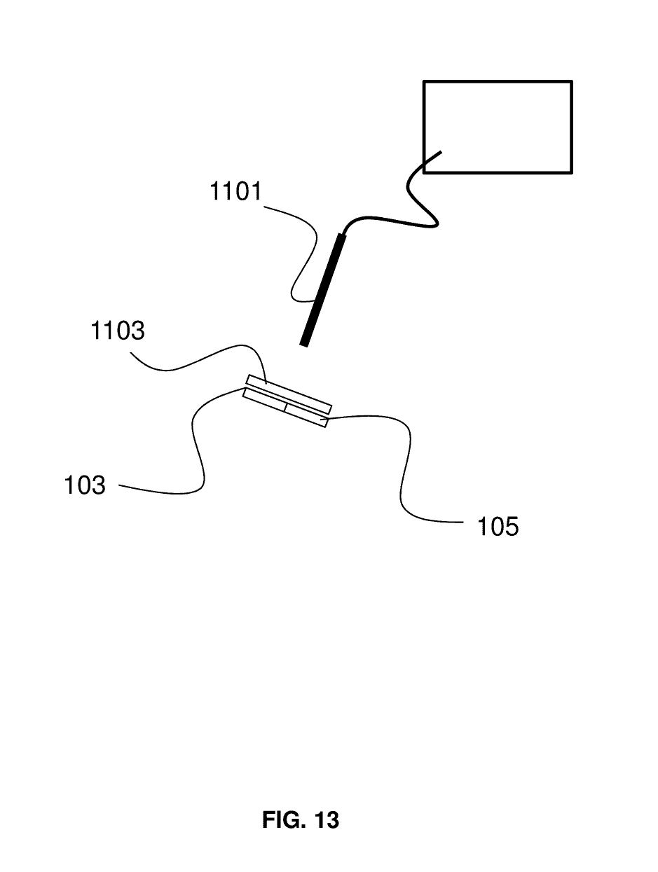

Lasers may be used to directly ablate the stratum corneum to provide the transdermal drug delivery benefits associated therewith and discussed above. Additionally, photomechanical waves, generated by lasers through confined ablation, have been shown to increase tissue permeability and enhance drug delivery by only transiently modifying the stratum corneum. See Lee, et al., Photomechanical Transdermal Delivery: The Effect of Laser Confinement, Lasers in Surgery and Medicine 28:344±347 (2001), the contents of which are incorporated herein in their entirety for all purposes. As described in Lee, lasers may be directed at a target above a solution reservoir, in turn above the tissue surface in order to propagate a photomechanical wave into the tissue. Id. Lasers are available, for instance, from Newport Corporation (Irvine, Calif.). Thus, in some embodiments, the invention provides a system for treating a viral infection that includes a laser 1001, a target 1103, and a solution comprising a vector encoding a gene for an enzyme that cuts target genetic material such as Cas9 103, and a gRNA that targets a latent virus and that has no match in the human genome, as shown in FIG. 13.

In various embodiments, chemical penetration enhancers may be used alone or in combination with one or more of the above methods of enhanced transdermal drug delivery. See Mitragotri, Synergistic Effect of Enhancers for Transdermal Drug Delivery, Pharm. Res. 17, 1354-1359, the contents of which are incorporated herein in their entirety for all purposes. Chemical penetration enhancers may include, for example, propylene glycol, oleic acid, DMSO, ethanol, linoleic acid, Azone, limonene, sodium lauryl sulfate, poly-ethylene glycol, isopropyl myristate, glycerol trioleate, and phosphate buffered saline.

Compositions of the invention may be delivered by any suitable method include subcutaneously, transdermally, by hydrodynamic gene delivery, topically, or any other suitable method. In some embodiments, the composition 101 is provided a carrier and is suitable for topical application to the human skin. The composition may be introduced into the cell in situ by delivery to tissue in a host. Introducing the composition into the host cell may include delivering the composition non-systemically to a local reservoir of the viral infection in the host, for example, topically.

A composition of the invention may be delivered to the affected area of the skin in an acceptable topical carrier such as any acceptable formulation that can be applied to the skin surface for topical, dermal, intradermal, or transdermal delivery of a medicament. The combination of an acceptable topical carrier and the compositions described herein is termed a topical formulation of the invention. Topical formulations of the invention are prepared by mixing the composition with a topical carrier according to well-known methods in the art, for example, methods provided by standard reference texts such as, REMINGTON: THE SCIENCE AND PRACTCE OF PHARMACY 1577-1591, 1672-1673, 866-885(Alfonso R. Gennaro ed.); Ghosh, T. K.; et al. TRANSDERMAL AND TOPICAL DRUG DELIVERY SYSTEMS (1997).

The topical carriers useful for topical delivery of the compound described herein can be any carrier known in the art for topically administering pharmaceuticals, for example, but not limited to, acceptable solvents, such as a polyalcohol or water; emulsions (either oil-in-water or water-in-oil emulsions), such as creams or lotions; micro emulsions; gels; ointments; liposomes; powders; and aqueous solutions or suspensions, such as standard ophthalmic preparations.

In certain embodiments, the topical carrier used to deliver the compositions described herein is an emulsion, gel, or ointment. Emulsions, such as creams and lotions are suitable topical formulations for use in accordance with the invention. An emulsion is a dispersed system comprising at least two immiscible phases, one phase dispersed in the other as droplets ranging in diameter from 0.1 μm to 100 μm. An emulsifying agent is typically included to improve stability.

In another embodiment, the topical carrier is a gel, for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are disclosed in REMINGTON: THE SCIENCE AND PRACTICE OF PHARMACY 1517-1518 (Alfonso R. Gennaro ed. 19th ed. 1995). Other suitable gels for use in the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002). Polymer thickeners (gelling agents) that may be used include those known to one skilled in the art, such as hydrophilic and hydro-alcoholic gelling agents frequently used in the cosmetic and pharmaceutical industries. Preferably the gelling agent comprises between about 0.2% to about 4% by weight of the composition. The agent may be cross-linked acrylic acid polymers that are given the general adopted name carbomer. These polymers dissolve in water and form a clear or slightly hazy gel upon neutralization with a caustic material such as sodium hydroxide, potassium hydroxide, or other amine bases.

In another preferred embodiment, the topical carrier is an ointment. Ointments are oleaginous semisolids that contain little if any water. Preferably, the ointment is hydrocarbon based, such as a wax, petrolatum, or gelled mineral oil.

In another embodiment, the topical carrier used in the topical formulations of the invention is an aqueous solution or suspension, preferably, an aqueous solution. Well-known ophthalmic solutions and suspensions are suitable topical carriers for use in the invention. The pH of the aqueous topical formulations of the invention are preferably within the range of from about 6 to about 8. To stabilize the pH, preferably, an effective amount of a buffer is included. In one embodiment, the buffering agent is present in the aqueous topical formulation in an amount of from about 0.05 to about 1 weight percent of the formulation. Tonicity-adjusting agents can be included in the aqueous topical formulations of the invention. Examples of suitable tonicity-adjusting agents include, but are not limited to, sodium chloride, potassium chloride, mannitol, dextrose, glycerin, and propylene glycol. The amount of the tonicity agent can vary widely depending on the formulation's desired properties. In one embodiment, the tonicity-adjusting agent is present in the aqueous topical formulation in an amount of from about 0.5 to about 0.9 weight percent of the formulation. Preferably, the aqueous topical formulations of the invention have a viscosity in the range of from 0.015 to 0.025 Pa·s (about 15 cps to about 25 cps). The viscosity of aqueous solutions of the invention can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose.

The topical formulations of the invention can include acceptable excipients such as protectives, adsorbents, demulcents, emollients, preservatives, antioxidants, moisturizers, buffering agents, solubilizing agents, skin-penetration agents, and surfactants. Suitable protectives and adsorbents include, but are not limited to, dusting powders, zinc sterate, collodion, dimethicone, silicones, zinc carbonate, aloe vera gel and other aloe products, vitamin E oil, allatoin, glycerin, petrolatum, and zinc oxide. Suitable demulcents include, but are not limited to, benzoin, hydroxypropyl cellulose, hydroxypropyl methylcellulose, and polyvinyl alcohol. Suitable emollients include, but are not limited to, animal and vegetable fats and oils, myristyl alcohol, alum, and aluminum acetate. Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin. Chlorine dioxide (ClO2), preferably, stabilized chlorine dioxide, is a preferred preservative for use with topical formulations of the invention. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use in the invention include, but are not limited to, acetate buffers, citrate buffers, phosphate buffers, lactic acid buffers, and borate buffers. Suitable solubilizing agents include, but are not limited to, quaternary ammonium chlorides, cyclodextrins, benzyl benzoate, lecithin, and polysorbates. Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone.

In some embodiments, the invention provides a system comprising a vector encoding a gene for an enzyme that cuts target genetic material such as Cas9 103, and a gRNA that targets a latent virus and that has no match in the human genome, a topical carrier, and a device such as one shown in FIGS. 5-13 configured to aid delivery of the topical carrier into the skin or other tissue.

In certain embodiments, compounds of the invention are conjugated to nano-systems for systemic therapy, such as liposomes, albumin-based particles, PEGylated proteins, biodegradable polymer-drug composites, polymeric micelles, dendrimers, among others. See Davis et al., 2008, Nanotherapeutic particles: an emerging treatment modality for cancer, Nat Rev Drug Discov. 7(9):771-782, incorporated by reference. Long circulating macromolecular carriers such as liposomes, can exploit the enhanced permeability and retention effect for preferential extravasation from tumor vessels. In certain embodiments, the complexes of the invention are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 April; 48(4):367-70. These cellular delivery systems may be introduced into the body transdermally through the methods described herein.

To deliver the Cas9 and sgRNAs, the invention may also provide for the use of hydrodynamic gene delivery. This technology controls hydrodynamic pressure in capillaries to enhance endothelial and parenchymal cell permeability (Hydrodynamic Gene Delivery: Its Principles and Applications, Molecular Therapy (2007) 15 12, 2063-2069). The first clinical test of hydrodynamic gene delivery in humans was reported at the 9th Annual Meeting of the American Society of Gene Therapy (Clinical Study with Hydrodynamic Gene Delivery into Hepatocytes in Humans). Hydrodynamic gene delivery avoids potential host immune response seen in AAV delivery (Prolonged susceptibility to antibody-mediated neutralization for adeno-associated vectors targeted to the liver.).

Hydrodynamic gene delivery can also be applied to liver transplant (Hydrodynamic plasmid DNA gene therapy model in liver transplantation). Injection volumes of 40-70% of the liver weight are found to be effective in gene delivery. Combination of hydrodynamic gene delivery with targeted endonuclease can potentially eliminate HBV from liver transplant, and provide more qualified organs.

The delivery of targeted endonuclease (e.g., Cas9+sgRNA) may be combined with conventional antiviral drugs, such as Lamivudine and Telbivudine. In such way, the viral load may be greatly reduced before endonuclease treatment to improve treatment efficacy.

For hydrodynamic gene delivery, a composition is delivered at a pressure sufficient to generate pores in the cells proximal to the blood vessel. Hydrodynamic or energy-enhanced transdermal gene delivery are used to deliver a nucleic acid such as a plasmid that preferably encodes an endonuclease enzyme. In a preferred embodiment, the enzyme is Cas9.

Cas9 (CRISPR associated protein 9) is an RNA-guided DNA endonuclease enzyme. Cas9 was found as part of the Streptococcus pyrogenes immune system, where it memorizes and later cuts foreign DNA by unwinding it to seek regions complementary to a 20 basepair spacer region of the guide RNA, where it then cuts. Cas9 can be used to make site-directed double strand breaks in DNA, which can lead to gene inactivation or the introduction of heterologous genes through non-homologous end joining and homologous recombination. Other exemplary tools for gene editing include zinc finger nucleases and TALEN proteins.

Cas9 can cleave nearly any sequence complementary to the guide RNA. Native Cas9 uses a guide RNA composed of two disparate RNAs that associate to make the guide—the CRISPR RNA (crRNA), and the trans-activating RNA (tracrRNA). Additionally or alternatively, Cas9 targeting may be simplified through the engineering of a chimeric single guide RNA.

Studies suggest that Cas9 contain RNase H and HNH endonuclease homologous domains which are responsible for cleavages of two target DNA strands, respectively. The sequence similar to RNase H has a RuvC fold (one member of RNase H family) and the HNH region folds as T4 Endo VII (one member of HNH endonuclease family). Previous works on Cas9 have demonstrated that HNH domain is responsible for complementary sequence cleavage of target DNA and RuvC is responsible for the non-complementary sequence. Methods and materials of the invention use a plasmid that includes a cas9 gene and at least one gene for a short guide RNA (sgRNA). The ssRNA is complementary to a portion of the viral genome.

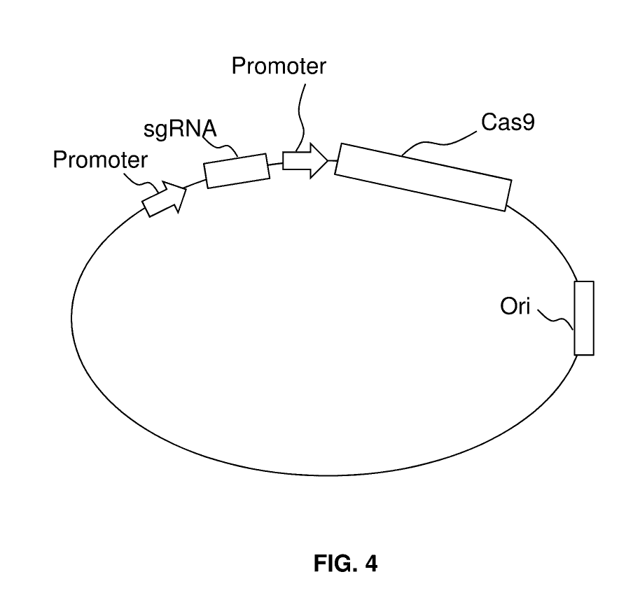

FIG. 4 diagrams a plasmid according to certain embodiments.

Where the viral genome is a hepatitis B genome, the plasmid may contain genes for one or more sgRNAs targeting locations in the hepatitis B genome such as PreS1, DR1, DR2, a reverse transcriptase (RT) domain of polymerase, an Hbx, and the core ORF. In a preferred embodiment, the one or more sgRNAs comprise one selected from the group consisting of sgHBV-Core and sgHBV-PreS1.

For hydrodynamic gene delivery, the composition may be delivered via an intravascular delivery catheter, e.g., by navigating a balloon catheter to the blood vessel at a target location in the subject, inflating the balloon, and delivering the composition via a lumen in the balloon catheter.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

In one embodiment, methods of the invention use gene delivery methods described above to target the hepatitis B virus (HBV). More than 40% of the human population has been infected with HBV, giving rise to 240 million chronic HBV carriers and ca. 620,000 HBV-associated deaths annually. Human Hepatitis B virus (HBV), which is the prototype member of the family Hepadnaviridae, is a 42 nm partially double stranded DNA virus, composed of a 27 nm nucleocapsid core (HBcAg), surrounded by an outer lipoprotein coat (also called envelope) containing the surface antigen (HBsAg). The virus includes an enveloped virion containing 3 to 3.3 kb of relaxed circular, partially duplex DNA and virion-associated DNA-dependent polymerases that can repair the gap in the virion DNA template and has reverse transcriptase activities. HBV is a circular, partially double-stranded DNA virus of approximately 3200 bp with four overlapping ORFs encoding the polymerase (P), core (C), surface (S) and X proteins. In infection, viral nucleocapsids enter the cell and reach the nucleus, where the viral genome is delivered. In the nucleus, second-strand DNA synthesis is completed and the gaps in both strands are repaired to yield a covalently closed circular DNA molecule that serves as a template for transcription of four viral RNAs that are 3.5, 2.4, 2.1, and 0.7 kb long. These transcripts are polyadenylated and transported to the cytoplasm, where they are translated into the viral nucleocapsid and precore antigen (C, pre-C), polymerase (P), envelope L (large), M (medium), S (small)), and transcriptional transactivating proteins (X). The envelope proteins insert themselves as integral membrane proteins into the lipid membrane of the endoplasmic reticulum (ER). The 3.5 kb species, spanning the entire genome and termed pregenomic RNA (pgRNA), is packaged together with HBV polymerase and a protein kinase into core particles where it serves as a template for reverse transcription of negative-strand DNA. The RNA to DNA conversion takes place inside the particles.

Numbering of basepairs on the HBV genome is based on the cleavage site for the restriction enzyme EcoR1 or at homologous sites, if the EcoR1 site is absent. However, other methods of numbering are also used, based on the start codon of the core protein or on the first base of the RNA pregenome. Every base pair in the HBV genome is involved in encoding at least one of the HBV protein. However, the genome also contains genetic elements which regulate levels of transcription, determine the site of polyadenylation, and even mark a specific transcript for encapsidation into the nucleocapsid. The four ORFs lead to the transcription and translation of seven different HBV proteins through use of varying in-frame start codons. For example, the small hepatitis B surface protein is generated when a ribosome begins translation at the ATG at position 155 of the adw genome. The middle hepatitis B surface protein is generated when a ribosome begins at an upstream ATG at position 3211, resulting in the addition of 55 amino acids onto the 5′ end of the protein.

ORF P occupies the majority of the genome and encodes for the hepatitis B polymerase protein. ORF S encodes the three surface proteins. ORF C encodes both the hepatitis e and core protein. ORF X encodes the hepatitis B X protein. The HBV genome contains many important promoter and signal regions necessary for viral replication to occur. The four ORFs transcription are controlled by four promoter elements (preS1, preS2, core and X), and two enhancer elements (Enh I and Enh II). All HBV transcripts share a common adenylation signal located in the region spanning 1916-1921 in the genome. Resulting transcripts range from 3.5 nucleotides to 0.9 nucleotides in length. Due to the location of the core/pregenomic promoter, the polyadenylation site is differentially utilized. The polyadenylation site is a hexanucleotide sequence (TATAAA) as opposed to the canonical eukaryotic polyadenylation signal sequence (AATAAA). The TATAAA is known to work inefficiently (9), suitable for differential use by HBV.

There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in-frame start (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. The function of the protein coded for by gene X is not fully understood but it is associated with the development of liver cancer. It stimulates genes that promote cell growth and inactivates growth regulating molecules.

HBV replicates its genome by reverse transcription of an RNA intermediate. The RNA templates is first converted into single-stranded DNA species (minus-strand DNA), which is subsequently used as templates for plus-strand DNA synthesis. DNA synthesis in HBV use oligoribonucleotides as primers for plus-strand DNA synthesis, which predominantly initiate at internal locations on the single-stranded DNA. The primer is generated via an RNase H cleavage that is a sequence independent measurement from the 5′ end of the RNA template. This 18 nt RNA primer is annealed to the 3′ end of the minus-strand DNA with the 3′ end of the primer located within the 12 nt direct repeat, DR1. The majority of plus-strand DNA synthesis initiates from the 12 nt direct repeat, DR2, located near the other end of the minus-strand DNA as a result of primer translocation. The site of plus-strand priming has consequences. In situ priming results in a duplex linear (DL) DNA genome, whereas priming from DR2 can lead to the synthesis of a relaxed circular (RC) DNA genome following completion of a second template switch termed circularization. It remains unclear why hepadnaviruses have this added complexity for priming plus-strand DNA synthesis, but the mechanism of primer translocation is a potential therapeutic target. As viral replication is necessary for maintenance of the hepadnavirus (including the human pathogen, hepatitis B virus) chronic carrier state, understanding replication and uncovering therapeutic targets is critical for limiting disease in carriers.

Guide RNA against PreS1 locates at the 5′ end of the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation. Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces.

FIG. 2 shows key parts in the HBV genome targeted by CRISPR guide RNAs.

FIG. 3 shows a gel resulting from an in vitro CRISPR assay against HBV. Lane 1, 3, 6: PCR amplicons of HBV genome flanking RT, Hbx-Core, and PreS1. Lane 2, 4, 5, and 7: PCR amplicons treated with sgHBV-RT, sgHBV-Hbx, sgHBV-Core, sgHBV-PreS1.

The materials of the invention are thus shown to fragment and HBV virus genome.

### Example 2

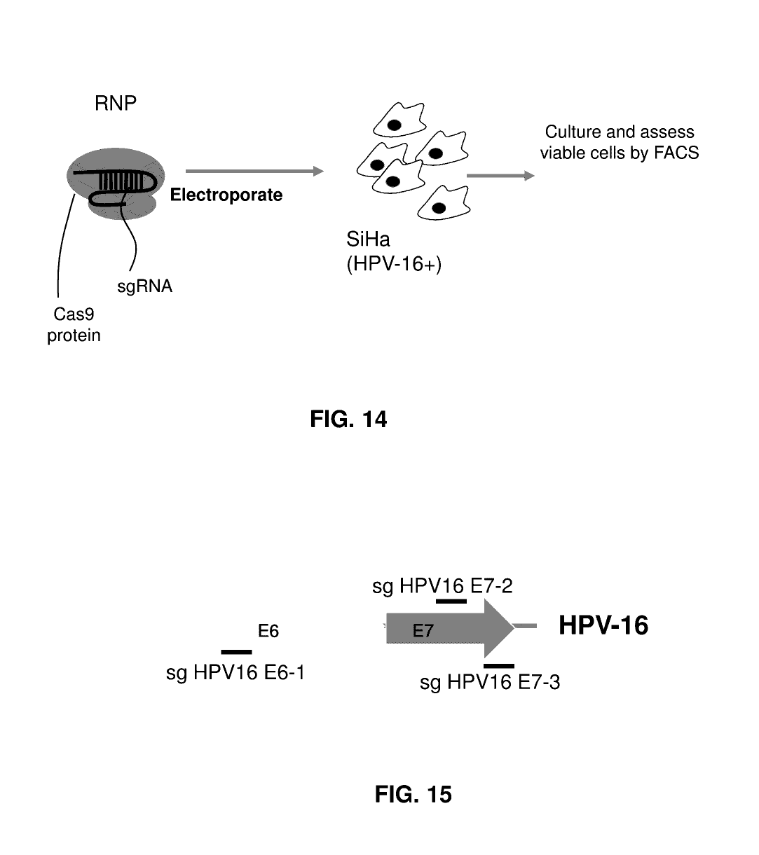

Electroporation may be used to introduce ribonucleoproteins (RNPs) to cells. HPV 16+ cancer cells were treated with HPV 16-specific CRISPR-Cas9 ribonucleoprotein (RNP) and found to kill HPV 16+ cancer cells. As illustrated in FIG. 14, an RNP comprising Cas9 protein and an sgRNA were introduced into SiHa HPV-16+ cells through electroporation. The cells were then cultured and viable cell counts were taken using fluorescence-activated cell sorting (FACS).

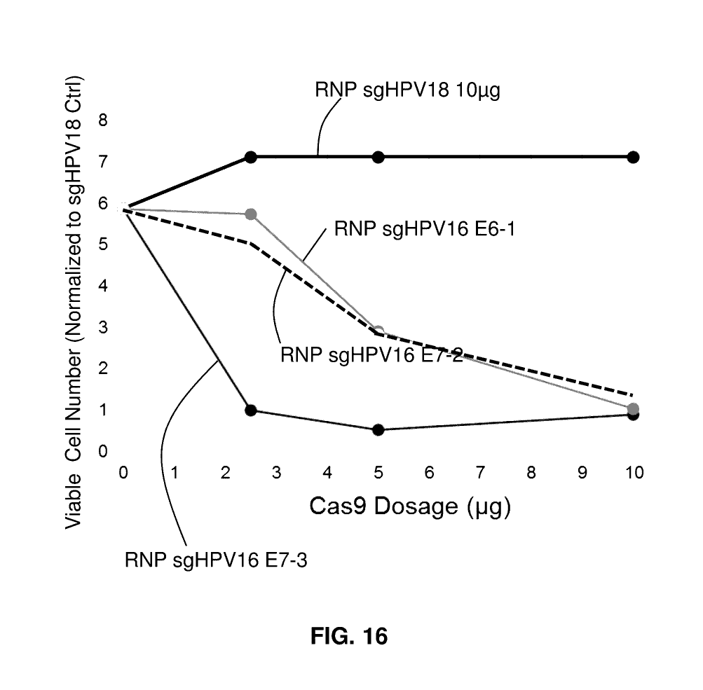

FIG. 15 shows target locations for various sgRNAs along the E6 and E7 genes of HPV-16. FIG. 16 illustrates cell counts after introduction by electroporation of RNPs with various sgRNAs with targets along the HPV-16 E6 and E7 genes as illustrated in FIG. 15. The cell counts are normalized to an sgHPV18 control and plotted by μg of Cas9 containing RNP dosage. Electroporation with RNPs comprising sgHPV16 E6-1, sgHPV16 E7-2, and sgHPV16 E7-3 all resulted in reduced cell counts when compared to the control as shown in FIG. 16.

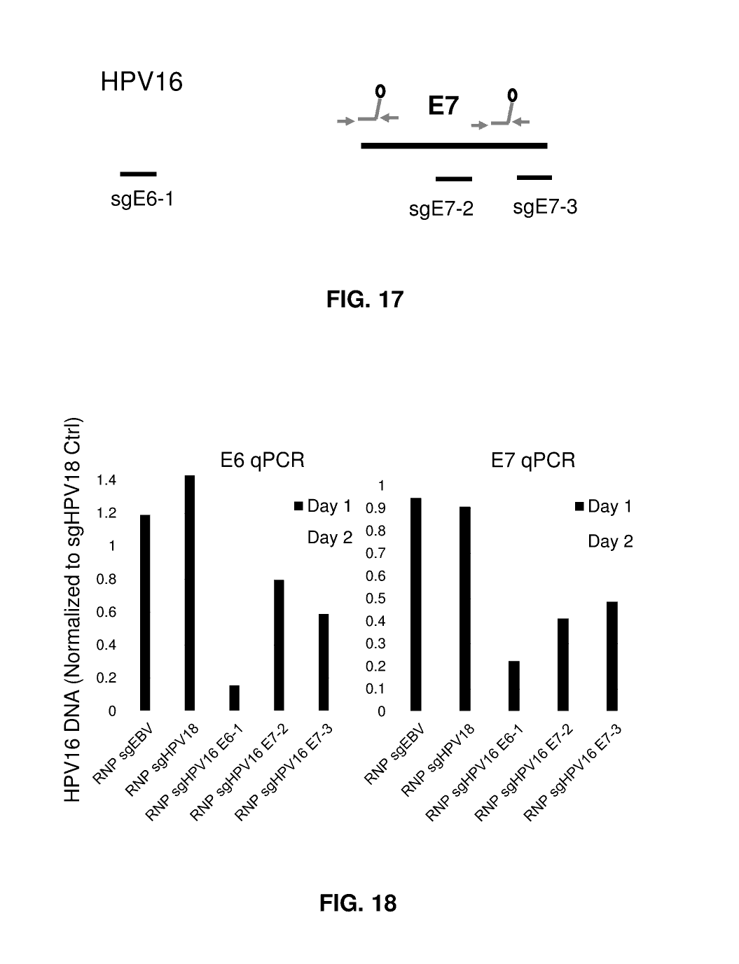

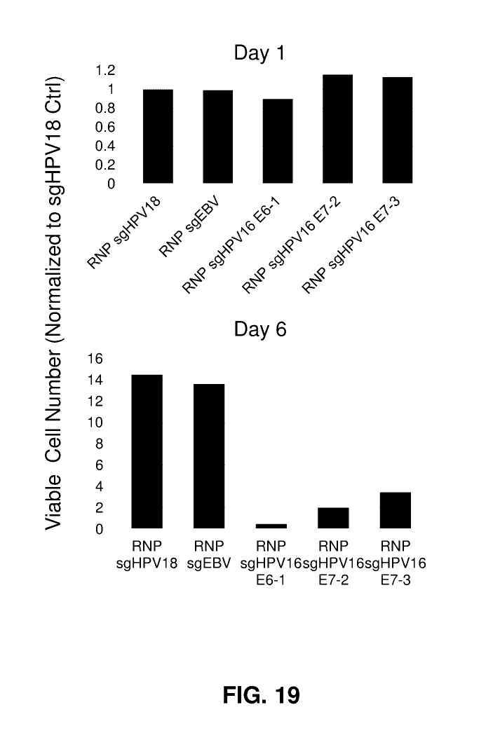

FIG. 17 illustrates target locations and quantitative PCR (qPCR) primer locations on the E6 and E7 genes of HPV-16. FIG. 18 shows qPCR results focusing on the E6 and E7 genes 1 and 2 days after treatment with various HPV 16-specific RNPs as normalized to an sgHPV18 RNP control. RNPs comprising sgHPV16 E6-1, sgHPV16 E7-2, and sgHPV16 E7-3 guide RNAs all exhibit cleavage of HPV 16 DNA at the E6 or E7 genes. Viable cell counts 1 and 6 days after treatment are shown in FIG. 19, normalized to the sgHPV18 control. Again, the three HPV 16 E6 and E7 targeting RNPs show the ability to reduce HPV 16 cell counts after electroporation of HPV 16+ cancer cells.

### Example 3

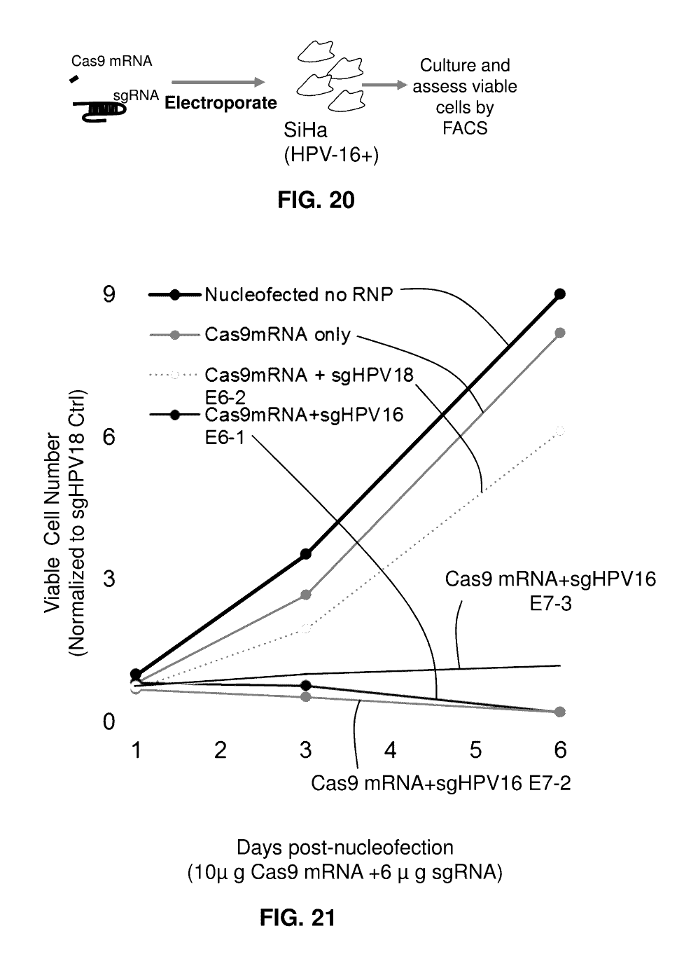

Electroporation may be used to introduce mRNA encoding an endonuclease along with a guide RNA. HPV 16+ cancer cells were treated by electroporation with HPV 16-specific sgRNA and Cas9 mRNA and found to kill HPV 16+ cancer cells. As illustrated in FIG. 20, an mRNA encoding Cas9 protein and an sgRNA were introduced into SiHa HPV-16+ cells through electroporation. The cells were then cultured and viable cell counts were taken using fluorescence-activated cell sorting (FACS).

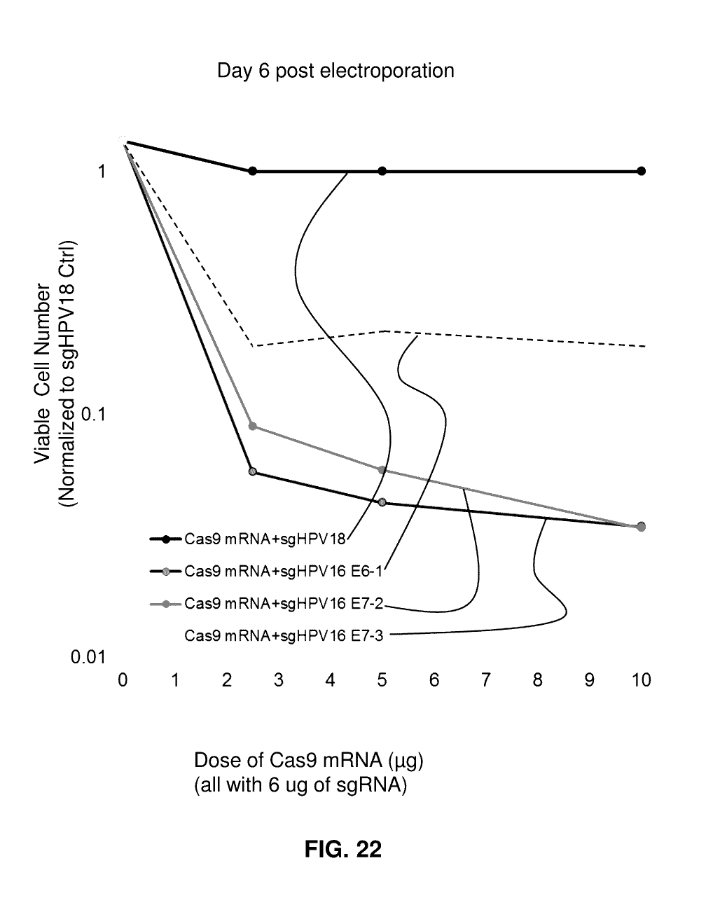

FIG. 21 shows normalized cell counts after 1, 3, and 6 days post-nucleofection with the various Cas9 mRNA and sgRNA combinations, all normalized to the sgHPV18 control. FIG. 22 shows cell counts for cells treated with 6 μg of the various sgRNA and a variety of Cas9 mRNA after 6 days, normalized to the sgHPV18 control. Both FIG. 21 and FIG. 22 show reduced cell counts in the cells nucleofected with HPV 16-specific sgRNAs and Cas9 mRna.

### Example 4

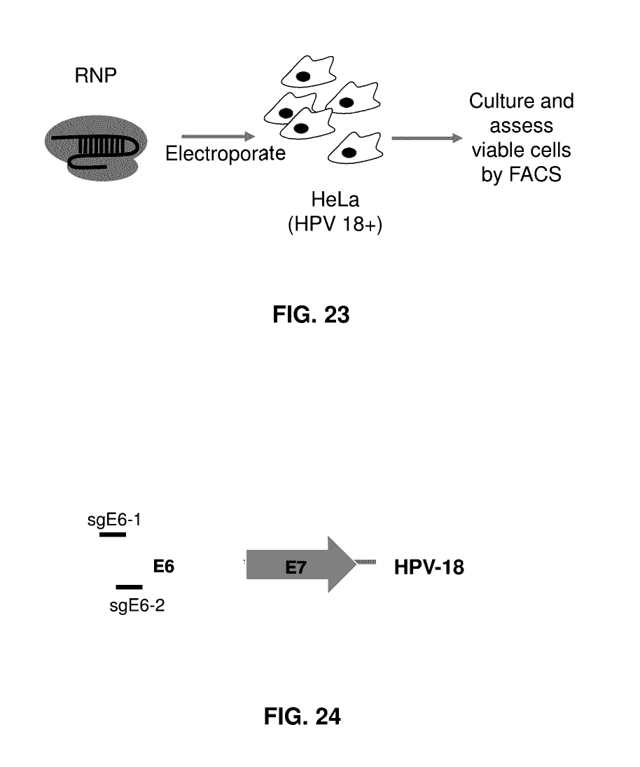

HPV 18+ cancer cells were treated with HPV 18-specific CRISPR-Cas9 ribonucleoprotein (RNP) and found to kill HPV 18+ cancer cells. As illustrated in FIG. 23, an RNP comprising Cas9 protein and an sgRNA were introduced into SiHa HPV-18+ cells through electroporation. The cells were then cultured and viable cell counts were taken using fluorescence-activated cell sorting (FACS).

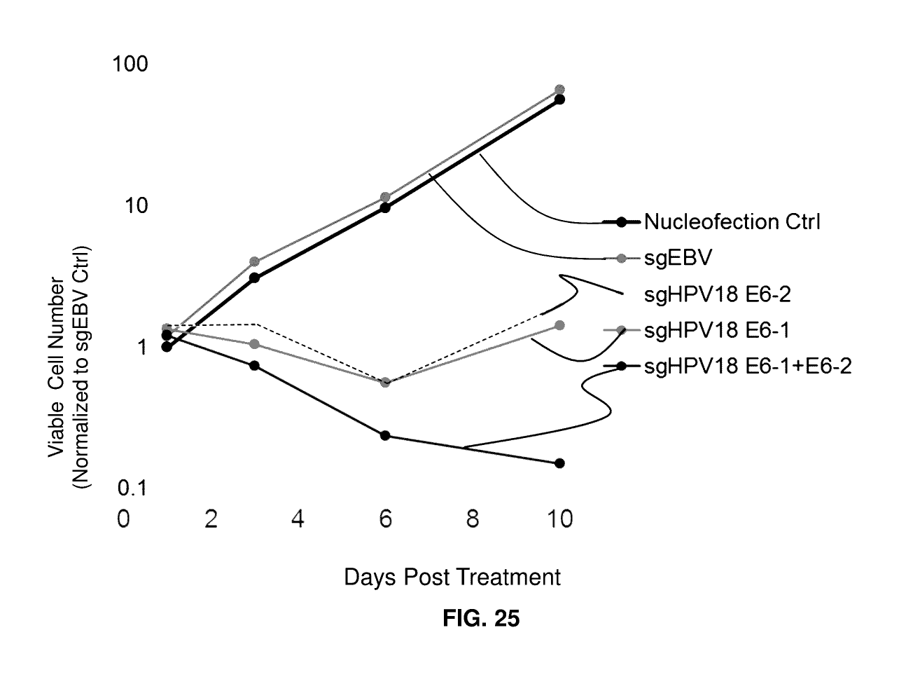

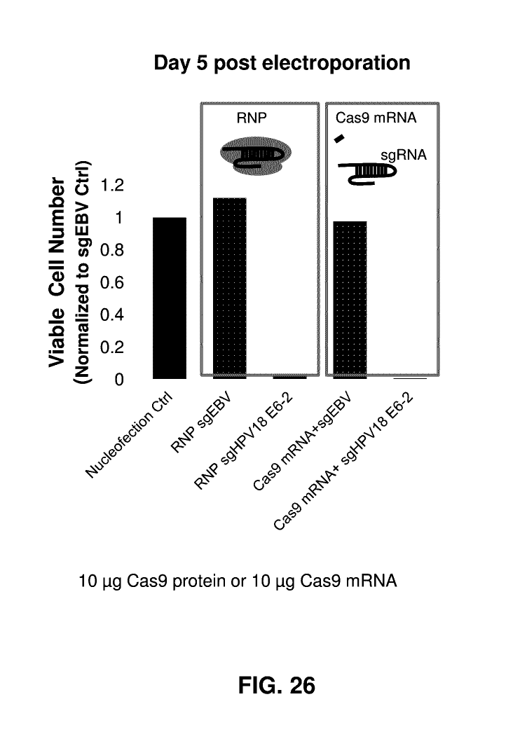

FIG. 24 shows target locations for various sgRNAs along the E6 gene of HPV-18. FIG. 25 illustrates cell counts after introduction by electroporation of RNPs with various sgRNAs targeting the HPV-18 E6 gene as illustrated in FIG. 24. The cell counts are normalized to an sgEBV control. Electroporation with RNPs comprising HPV 18-specific RNPs all resulted in reduced cell counts when compared to the control as shown in FIG. 25. FIG. 26 shows a viable cell count comparison for HPV-18+ cancer cells 5 days post electroporation with sgHPV18E6-2/Cas9 in RNP format or in mRNA/sgRNA format. Both methods clearly resulted in reduced cell counts.

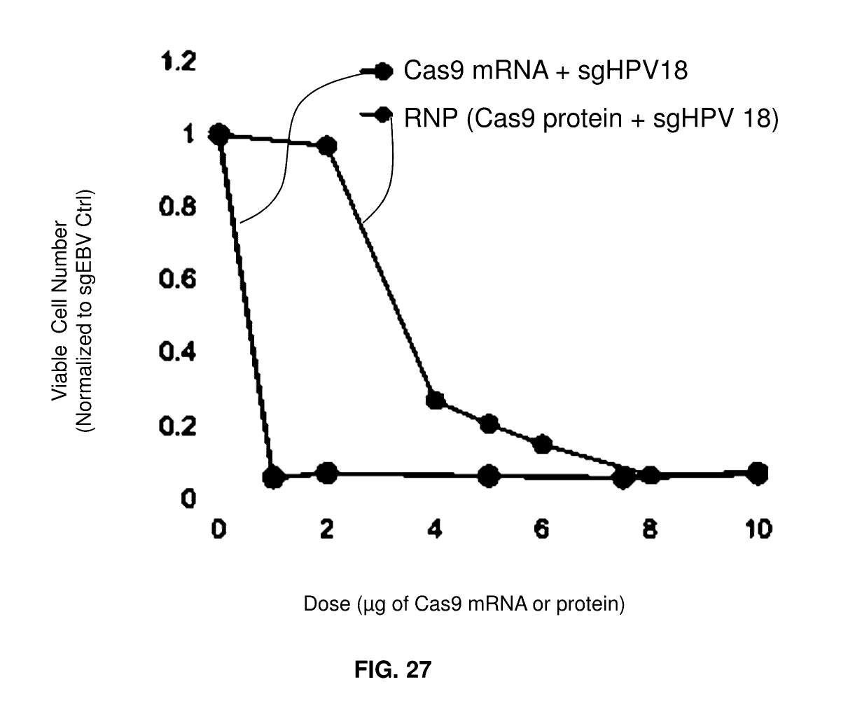

FIG. 27 shows a comparison of viable cell counts in mRNA and RNP treated cells by μg dose of Cas9 mRNA or protein. The mRNA treatment greater reduction than RNP treatment at lower dosages and the treatment methods produced similar results at increased dosages.

### Example 5

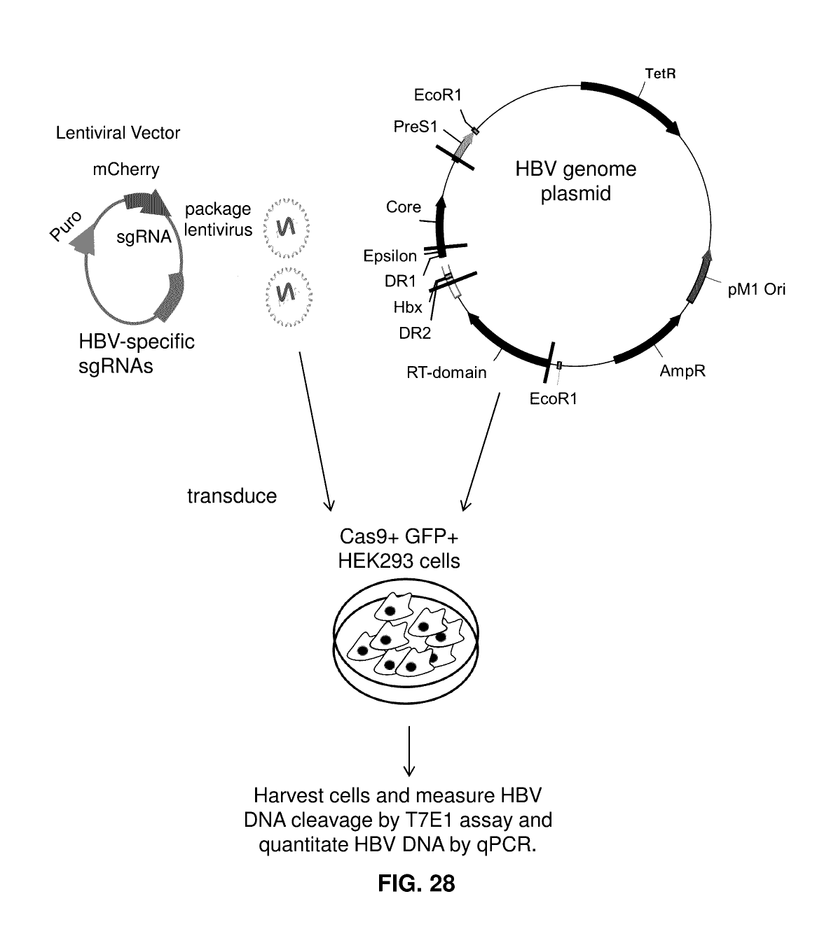

Embodiments of the invention may be used to introduce nucleic acid encoding guided endonucleases targeting the DNA of various viruses, such as HBV. Cas9 in coordination with various HBV-specific guide RNAs has been shown to reduce viral DNA load in cells through targeted cleavage at certain cites in the viral genome. FIG. 28 illustrates an HBV episomal DNA cell model. Cas9+ GFP+ HED293 cells were transfected with an HBV genome plasmid as shown. HBV-specific sgRNAs were then introduced through transduction using a lentiviral vector. The cells were then harvested an HBV DNA cleavage was measured by T7E1 assay and HBV DNA was measured by qPCR.

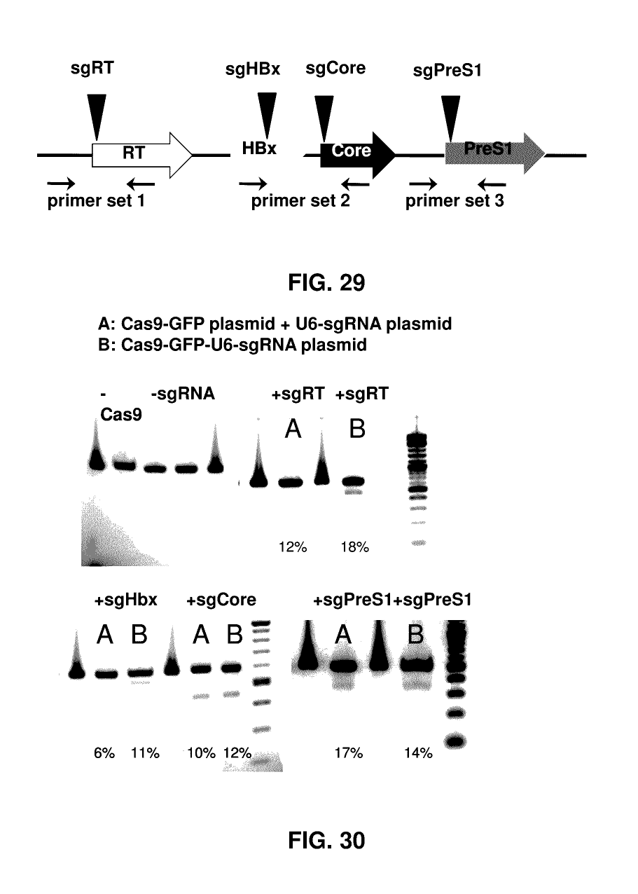

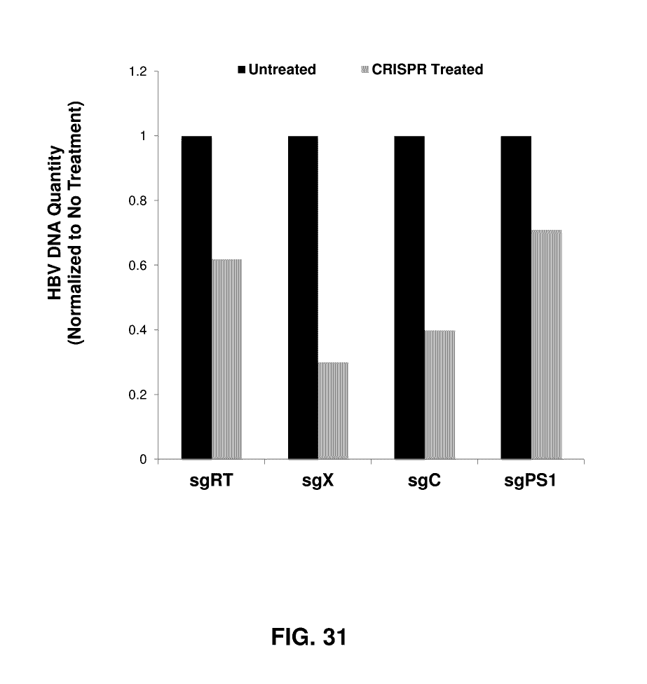

FIG. 29 shows the target locations on the HBV genome of various sgRNAs used in the model along with the location of primer set targets used to assess HBV DNA cleavage. FIG. 30 shows the results of gel electrophoresis indicating cleavage of HBV DNA in cells transduced with sgRT RNA, sgHBx RNA, sgCore RNA, and sgPreS1 RNA. FIG. 31 shows HBV DNA quantity determined by qPCR in untreated cells and cells treated with HBV-specific sgRNAs and Cas9. Each of the four tested sgRNAs exhibited reduced HBV DNA quantity when compared to untreated cells. The results illustrated in FIGS. 30 and 31 are from unsorted cells 2 days post treatment.

What is claimed is:

1. A method for disrupting target genetic material from a subject, the method comprising:
delivering a composition comprising a nucleic acid to tissue by applying an energy to the tissue to increase permeability of the tissue thereby causing the nucleic acid to enter cells of the tissue, wherein the nucleic acid encodes a programmable nuclease.

2. The method of claim 1, wherein the programmable nuclease is Cas9.

3. The method of claim 1, wherein the energy is applied through electroporation.

4. The method of claim 1, wherein the applied energy is ultrasound.

5. The method of claim 2, wherein the nucleic acid is a plasmid comprising a cas9 gene and at least one gene for a short guide RNA (sgRNA).

6. The method of claim 5, wherein the target genetic material is viral.

7. The method of claim 6, wherein the sgRNA is complementary to a portion of a viral genome and has no match in a human genome.

8. The method of claim 7, wherein the viral genome is a hepatitis B genome and the plasmid contains genes for one or more sgRNAs targeting locations in the hepatitis B genome.

9. The method of claim 8, wherein the one or more sgRNAs target locations in the hepatitis B genome selected from PreS1, DR1, DR2, a reverse transcriptase (RT) domain of polymerase, an Hbx, and the core ORF.

10. The method of claim 9, wherein the one or more sgRNAs comprise one selected from the group consisting of sgHBV-Core and sgHBV-PreS1.

11. The method of claim 1, wherein the target genetic material is genome of a virus, and wherein the nucleic acid is a plasmid comprising a cas9 gene and at least one sgRNA targeting the genome of the virus.

12. The method of claim 11, wherein the plasmid further includes a viral origin of replication.

13. The method of claim 11, wherein the virus is hepatitis B and the at least one sgRNA selected from the group consisting of sgHBV-RT, sgHBV-Hbx, sgHB V-Core, and sg-HBV-PerS1.

14. The method of claim 1, wherein the nucleic acid comprises mRNA comprising a 5′ cap.

15. The method of claim 1, wherein the enzyme is a transcription activator-like effector nuclease (TALEN).

16. A method for treating a subject, the method comprising:
delivering a composition comprising a programmable nuclease to a tissue by applying energy to the tissue to increase permeability of the tissue thereby causing the programmable nuclease to enter cells of the tissue.

17. The method of claim 16, wherein the programmable nuclease is an RNA-guided nuclease complexed with a guide RNA as a ribonucleoprotein (RNP).

18. The method of claim 17, wherein the programmable nuclease is Cas9.

19. The method of claim 17, wherein applying the energy comprises electroporation of the tissue.

20. The method of claim 16, wherein the applied energy comprises ultrasound energy.
